(19) United States
(12) Patent Application Publication
(10) Pub. No.: US 2013/0096097 A1
(43) Pub. Date: Apr. 18, 2013

(54) PHARMACEUTICAL MULTIMERIC PARTICLES, AND MANUFACTURING METHOD FOR SAME

(71) Applicants: Hitoshi Kasai, Sendai-shi (JP); Hachiro Nakanishi, Sendai-shi (JP); Koichi Baba, Mino-shi (JP); Hidetoshi Oikawa, Sendai-shi (JP); Tastuya Murakami, Kyoto-shi (JP); Hiroshi Imahori, Kyoto-shi (JP); Misturu Hashida, Kyoto-shi (JP); Isamu Oh, Osaka (JP)

(72) Inventors: Hitoshi Kasai, Sendai-shi (JP); Hachiro Nakanishi, Sendai-shi (JP); Koichi Baba, Mino-shi (JP); Hidetoshi Oikawa, Sendai-shi (JP); Tastuya Murakami, Kyoto-shi (JP); Hiroshi Imahori, Kyoto-shi (JP); Misturu Hashida, Kyoto-shi (JP); Isamu Oh, Osaka (JP)

(73) Assignee: JAPAN SCIENCE AND TECHNOLOGY AGENCY, Kawaguchi-shi, Saitama (JP)

(21) Appl. No.: 13/702,205

(22) PCT Filed: Jun. 7, 2011
(86) PCT No.: PCT/JP2011/063084

§ 371 Date: Dec. 5, 2012

(30) Foreign Application Priority Data
Jun. 11, 2010 (JP) 2010-133486

(51) Int. Cl. A61K 9/10

(52) U.S. Cl. CPC A61K 9/10
USPC 514/180; 546/48; 514/283; 552/509

(57) ABSTRACT

[Problem] The purpose of the present invention is to provide organic particles containing pharmaceutical particles of which the particles are small and the particle size distribution is narrow, and a manufacturing method for the same.

[Solution] Provided are pharmaceutical multimeric particles of which the particles are small and the particle size distribution is narrow and which are characterized in being obtained by pouring into water a solution of a pharmaceutical multimer dissolved in a water-miscible organic solvent, and a manufacturing method for the pharmaceutical multimeric particles. Pharmaceutical dimeric particles thereof are characterized in being obtained by pouring into water a solution of a compound represented by general formula (I) dissolved in a water-miscible organic solvent.

### TECHNICAL FIELD

The present invention relates to pharmaceutical multimeric particles and a manufacturing method for the same.

### BACKGROUND OF THE INVENTION

Organic particles are used in a variety of fields based on the properties such as high chemical activity, specific electronic state, good dispersion stability and the like.

For example, in the field of pharmaceuticals, poorly-soluble drugs are often used as aqueous suspension agent wherein pharmaceutical particles are dispersed in water. Since aqueous suspension agents exert the medicinal effect by gradually eluting the drug component from particles in the body, it is required that the particle size of the pharmaceutical particles is as small as possible in order to exert the medicinal effect efficiently.

In addition, organic pigments wherein poorly-soluble organic dyes are microparticulated are used for various purposes such as toner for electrophotography, ink-jet ink, color filter, paint, printing ink, and the like. Regarding these organic pigments, it is also desired from the viewpoint of improving in various qualities such as color reproducibility, image quality, dispersion stability, and the like that the particles are smaller and have uniform particle size.

Organic particles exert a superior performance in various uses including these by making them smaller, therefore development of technology is proceeding with nano-microparticulation of organic compounds.

Traditionally, particles of poorly-soluble drugs and pigments are produced by mechanically pulverizing coarse particles with ball mill, etc. However, it is difficult to decrease the particle size to nanometer scale, also the particle size varies greatly.

Meanwhile, when a drug is administered to human body, a poorly-soluble drug is often used as an aqueous suspension agent wherein the drug is dispersed in water.

Having a property that the fine particulate drug component dispersed in water remains in the body, aqueous suspension agents are effective in exerting medicinal effects locally or over a long time, and used as oral preparation, dermatological topical preparation, nasal topical preparation, ophthalmologic topical preparation, and the like according to the purpose.

Aqueous suspension agents are usually produced by dispersing microparticulated drug into water with using a surfactant and the like. However the particle size of the drug obtained by pulverization is relatively large and the particle size varies greatly, thus there was a problem that large particle components settle out during long-term storage or by a rapid change of environment such as temperature. In addition, due to the variation of particle size, the quality as aqueous suspension agents is subject to variation, therefore there was a problem that even in the drug produced by the same manufacturing method, the retention time in some cases is too short to exert its effect, while in other cases it is so long that there is a threat of producing side effects. Thus methods for producing pharmaceutical particles wherein the particle size is small and the particle size distribution is narrow are desired.

On the other hand, Camptothecin that is an anticancer agent and specific derivatives thereof have been known (US2007041093, US20100233190, WO2008/034124, U.S. Pat. No. 6,011,042, WO98/035940). Camptothecin and most of its derivatives are extremely insoluble in water, so clinical application of this drug is constrained. As water-soluble Camptothecin derivatives, 9-dimethylaminomethyl-10-hydroxycamptothecin (Topotecan), 7-[(4-methylpiperazino)methyl]-10,11-ethylenedioxycamptothecin, 7-[(4-methylpiperazino)methyl]-10,11-methylenedioxycamptothecin, and 7-ethyl-10-[4-(1-piperidino)-1-piperidino]carbonyloxycamptothecin (CPT-11) are exemplified. The use of CPT-11 (Irinotecan/Camptosar, Resistered trade mark) for human was approved by the U.S. Food and Drug Administration in June 1996.

In 1984, U.S. Pat. No. 4,473,692 by Miyasaka et al. disclosed that the active substance of CPT-11 is 10-hydroxy-7-ethylcamptothecin (SN-38). The water-solubility of SN-38 is extremely poor, so it was not directly administered to human cancer patients. In recent years, it is reported that in human patients, SN-38 is further metabolized to form inactivated glucuronide species. NK012 that is a polymer micelle preparation of SN-38 is being developed, but micellar nanoparticles have various problems, so its practical use is questioned.

In addition, clinical development of liposome preparation (LE-SN38) of SN-38 is carried out, however it cannot pass the primary tumor response end point yet. Therefore desired is the method for development of new drug having SN-38 as a medicinally effective component and using novel DDS.

On the other hand, the present inventors have developed a novel method for reprecipitation technique to produce a dispersion solution of nanometer scale particles (JP-A 6-079168). This is to produce a dispersion solution of ultrafine particles of organic material by pouring a solution of said organic material dissolved in good solvent into poor solvent. According to this method, it is possible to produce a dispersion solution of fine particles of various materials.

### PRIOR ART

### Patent Document

patent document 1: US2007041093
patent document 2: US20100233190
patent document 3: WO2008/034124
patent document 4: U.S. Pat. No. 6,011,042
patent document 5: WO98/035940
patent document 6: U.S. Pat. No. 4,473,692
patent document 7: JP6-079168A

### DISCLOSURE OF INVENTION

### Problems to be Resolved by the Invention

According to this method, it is possible to manufacture a dispersion solution of fine crystals of various substances. However, in some kind of drugs, this method cannot give particles having satisfactory particle size and particle size distribution, therefore a method applicable to various kinds of drugs is desired. Depending on a combination of organic compound and poor solvent, there was a case where it is difficult to obtain particles having sufficiently small particle size.

Therefore, the object of the present invention is to provide organic particles containing pharmaceutical particles having small particle size and narrow particle size distribution and a manufacturing method for the same.

### Means of Solving the Problems

The present inventors have intensively investigated, and as the result, have found that the above-mentioned problems can be solved by using pharmaceutical multimer wherein plural drugs are linked via biodegradable substituents, and finally completed the present invention.

Further, the present inventors have found that the above-mentioned problems can be solved by using, as a crystal nucleating agent, organic compound plurally having the same main skeleton as that of the organic compound forming particles, and finally completed the present invention.

That is, the first aspect of the present invention provides particles of pharmaceutical multimer (pharmaceutical multimeric particles), wherein the pharmaceutical multimer is characterized by being formed by intermolecular dehydration condensation between a of plural drugs represented by the following general formula (1), and being hydrolyzed in the living body to plural drugs represented by the above-mentioned general formula (1), and said pharmaceutical multimeric particles are characterized by being obtained by pouring a solution of the pharmaceutical multimer dissolved in a water-miscible organic solvent into water.

[Chemical Formula (1)]

Aa)ₖ (1)

wherein, in the general formula (1), A is a k-valent organic group, k is an integer of 2 or more, and a is a monovalent substituent. k of a may be the same or different each other.

In addition, the second aspect of the present invention provides pharmaceutical multimeric particles of a drug represented by the following general formula (2), that are characterized by being obtained by pouring a solution of the compound represented by the following general formula (3) dissolved in a water-miscible organic solvent into water.

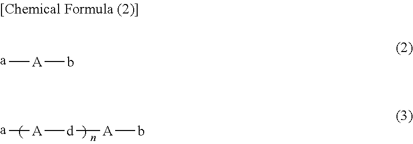

wherein, in the general formula (2) and general formula (3), A is a divalent organic group, a and b are each a substituent selected from hydroxyl group, amino group, carboxyl group, thiol group and hydrogen atom (provided that limited to the hydrogen atom on secondary amine). In the general formula (3), n is an integer of 1 or more, and d is a divalent substituent wherein by the hydrolysis of d in the living body, the compound represented by the general formula (3) is converted to n+1 of drugs represented by the general formula (2).

Further, the third aspect of the present invention provides pharmaceutical multimeric particles of a drug represented by the following general formula (4), that are characterized by being obtained by pouring a solution of the compound represented by the following general formula (5) dissolved in a water-miscible organic solvent into water, and solves the above-mentioned problem.

[Chemical Formula (3)]

a-A (4)

Bd-A)ₘ (5)

wherein, in the general formula (4) and general formula (5), A is a monovalent organic group. In the general formula (4), a is a substituent selected from hydroxyl group, amino group, carboxyl group, thiol group and hydrogen atom (provided that, limited to the hydrogen atom on secondary amine). In the general formula (5), m is an integer of 2 or more, B is a m-valent substituent, and d is a divalent substituent wherein by hydrolysis of d in the living body, the compound represented by the general formula (5) is converted to the compound represented by B(-f)m wherein f is a substituent selected from hydroxyl group, amino group, carboxyl group and thiol group, and m of drug represented by the general formula (4).

In the above-mentioned third aspect, the compound represented by B(-f)m is preferably any one of succinic acid, fumaric acid, tartaric acid, malic acid, ascorbic acid, citric acid, lactic acid, amino acid, maleic acid, malonic acid, adipic acid, triethanolamine, trometamol and glycerin.

In addition, in the above-mentioned third aspect, d is preferably any one of groups of —NHCO—, —CONH—, —COO— and —OCOO—.

Further, the fourth aspect of the present invention provides pharmaceutical multimeric particles, that are characterized by being obtained by pouring a solution of the compound represented by the following general formula (I) dissolved in a water-miscible organic solvent into water.

[Chemical Formula (4)]

D-R-L-R-D (I)

wherein, D represents an anticancer agent, L represents a group selected from a group consisting of an optionally substituted alkyl, an optionally substituted cycloalkyl, an optionally substituted alkenyl, an optionally substituted alkynyl and an optionally substituted aryl, R represents a group selected from a group consisting of —C(═O)—, —C(═O)O—, —C(═O)NR1-, —C(═O)OC(═O)—, —C(═O)OC(═O)O—, —O—, —NHR1-, —S(═O)—, —SO2-, —SO(NR1)-, —SO2NR1, —P(═O)(OR1)O—, —P(═O)(NR)O— and alkylene, R1 represents a hydrogen, alkyl, an optionally substituted cycloalkyl or an optionally substituted aryl.

In the above-mentioned fourth aspect, preferably D is Camptothecin derivatives, L is an optionally substituted alkyl or an optionally substituted alkenyl, R is —C(═O)—, —C(═O)NR1- or alkylene, and R1 is a hydrogen.

More preferably D is SN-38, L is an alkyl or an alkenyl, R is —C(═O)—, —C(═O)NH— or alkylene.

Furthermore, in the above-mentioned first, second, third and fourth aspect, as drugs to be multimerized, preferred are poorly water-soluble drugs having solubility of 1.0 g/L or less in 25° C. water.

The fifth aspect of the present invention provides a manufacturing method of pharmaceutical multimeric particles to solve the above-mentioned problem, wherein the manufacturing method of pharmaceutical multimeric particles is characterized by pouring a solution of pharmaceutical multimer dissolved in a water-miscible organic solvent into water, and said pharmaceutical multimer is characterized by being formed by intermolecular condensation between a of plural drugs represented by the following general formula (1), and being converted in the living body to plural drugs represented by the general formula (1).

[Chemical Formula (5)]

Aa)ₖ (1)

wherein, in the general formula (1), A is a k-valent organic group, k is an integer of 2 or more, and a is a monovalent substituent. k of a may be the same or different each other.

The sixth aspect of the present invention provides a manufacturing method of pharmaceutical multimeric particles of a drug represented by the following general formula (4) to solve the above-mentioned problem, which is characterized by pouring a solution of the compound represented by the following general formula (5) dissolved in a water-miscible organic solvent into water.

[Chemical Formula (6)]

a-A (4)

Bd-A)ₘ (5)

wherein, in the general formula (4) and general formula (5), A is a monovalent organic group. In the general formula (4), a is a substituent selected from hydroxyl group, amino group, carboxyl group, thiol group and hydrogen atom (provided that, limited to the hydrogen atom on secondary amine). In the general formula (5), m is an integer of 2 or more, B is a m-valent substituent, and d is a divalent substituent wherein by hydrolysis of d in the living body, the compound represented by the general formula (5) is converted to the compound represented by B(-f)m wherein f is a substituent selected from hydroxyl group, amino group, carboxyl group and thiol group, and m of drug represented by the general formula (4).

The seventh aspect of the present invention provides organic particles of the compound represented by the general formula (6) to solve the above-mentioned problem, which are characterized by being obtained by pouring a solution of the compound represented by the following general formula (6) and a crystal nucleating agent that is a compound having plural partial structure A possessed by the compound represented by the general formula (6) dissolved in organic solvent into a solvent which is a poor solvent for the compound represented by the general formula (6) and the crystal nucleating agent and is compatible with the organic solvent, and are characterized in that the crystal nucleating agent is used with an amount wherein the total amount of partial structure A possessed by the crystal nucleating agent is 0.0001-50 mole % to the compound represented by the general formula (6), and the total atom number of all partial structure A in the crystal nucleating agent molecule is more than the total atom number of the other parts.

[Chemical Formula (7)]

A′a′)_{k′} (6)

wherein, in the general formula (6), A′ is a k′-valent organic group, k′ is an integer of 1 or more, and a′ is hydrogen atom or a monovalent substituent. k′ of a′ may be the same or different each other. In addition, in the general formula (6), the atom number is in relation of (atom number possessed by partial structure A′>atom number possessed by the other parts).

In addition, the eighth aspect of the present invention provides organic particles of the compound represented by general formula (7) to solve the above-mentioned problem, which are characterized by being obtained by pouring a solution of the compound represented by the following general formula (7) and a compound represented by the following general formula (8) wherein the total amount of partial structure A is 0.0001-50 mole % to said compound represented by the general formula (7) dissolved in organic solvent into a solvent that is a poor solvent for the compounds represented by the general formula (7) and general formula (8) and is compatible with the organic solvent.

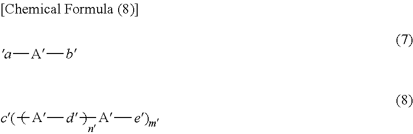

wherein, in the general formula (7) and general formula (8), A′ is a divalent organic group, a′, b′ and e′ are each independently a hydrogen atom or a monovalent substituent, c′ is a hydrogen atom or a m-valent substituent, d′ is a divalent linking group or a simple bond, m′ is an integer of 1 or more, n′ is an integer of 1 or more. m′ of e′ and n′, and m′×n′ of d may be each the same or different, but all of A that are present in the general formula (7) and general formula (8) have the same structure. In addition, in the general formula (7), the atom number is in relation of A′>(a′+b′).

In this aspect, it is preferred that A′ in general formula (7) includes an aromatic and/or non-aromatic cyclic organic group, and a′ and b′ don't include an aromatic and/or non-aromatic cyclic organic group.

Further in this aspect, the compound represented by the general formula (8) is preferably a compound represented by the following general formula (9).

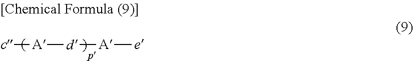

wherein, in the general formula (9), A′, d′ and e′ are each the same as defined in general formula (8), c″ is a hydrogen atom or a monovalent substituent and p′ is an integer of 1 or more. Provided that, all of A that are present in the general formula (7) and general formula (9) have the same structure, and p′ of d′ are all the same structure.

Further in this aspect, preferably a′ and b′ in general formula (7) are each independently a monovalent substituent having atom number of main chain 1 to 3 except hydrogen atom, or a hydrogen atom, and d′ in the general formula (8) or general formula (9) is a divalent linking group having atom number of main chain 1 to 3 except hydrogen atom, or simply a bond, and furthermore preferably a′ and b′ in general formula (7) are each any one of carboxyl group, hydroxyl group, amino group and halogen atom.

In addition, the organic compound represented by the general formula (9) is preferably a multimer of the organic compound represented by the general formula (7), that is formed by intermolecular condensation between a′ and b′ of the organic compound represented by the general formula (7).

The ninth aspect of the present invention is a manufacturing method of organic particles of the compound represented by the general formula (6), which is characterized by pouring a solution of the compound represented by the following general formula (6) and a crystal nucleating agent that is a compound having plural partial structure A possessed by the compound represented by the general formula (6) dissolved in organic solvent into a solvent that is a poor solvent for the compound represented by the general formula (6) and the crystal nucleating agent and is compatible with the organic solvent, and the manufacturing method of organic particles of the compound represented by the general formula (6) characterized in that the crystal nucleating agent is used with an amount wherein the total amount of partial structure A possessed by the crystal nucleating agent is 0.0001-50 mole % to the compound represented by the general formula (1) and the total atom number of all partial structure A in the crystal nucleating agent molecule is more than the total atom number of the other parts, is provided to solve the above-mentioned problem.

[Chemical Formula (10)]

A′a′)_{k′} (6)

wherein, in the general formula (6), A′ is a k′-valent organic group, k′ is an integer of 1 or more and a′ is a hydrogen atom or a monovalent substituent. k′ of a′ may be the same or different each other. In addition, in the general formula (6), the atom number is in relation of (atom number possessed by partial structure A′>atom number possessed by the other parts).

In addition, the tenth aspect of the present invention provides a manufacturing method of pharmaceutical multimeric particles, which is characterized by pouring a solution of the compound represented by the following general formula (I) dissolved in a water-miscible organic solvent into water.

[Chemical Formula (11)]

D-R-L-R-D (I)

wherein, D represents an anticancer agent, L represents a group selected from a group consisting of an optionally substituted alkyl, an optionally substituted cycloalkyl, an optionally substituted alkenyl, an optionally substituted alkynyl and an optionally substituted aryl, R represents a group selected from a group consisting of —C(═O)—, —C(═O)O—, —C(═O)NR1-, —C(═O)OC(═O)—, —C(═O)OC(═O)O—, —O—, —NHR1-, —S(═O)—, —SO2-, —SO(NR1)-, —SO2NR1, —P(═O)(OR1)O—, —P(═O) (NR)O— and alkylene, R1 represents a hydrogen, alkyl, an optionally substituted cycloalkyl or an optionally substituted aryl.

Further, the eleventh aspect of the present invention provides the following pharmaceutical dimers.

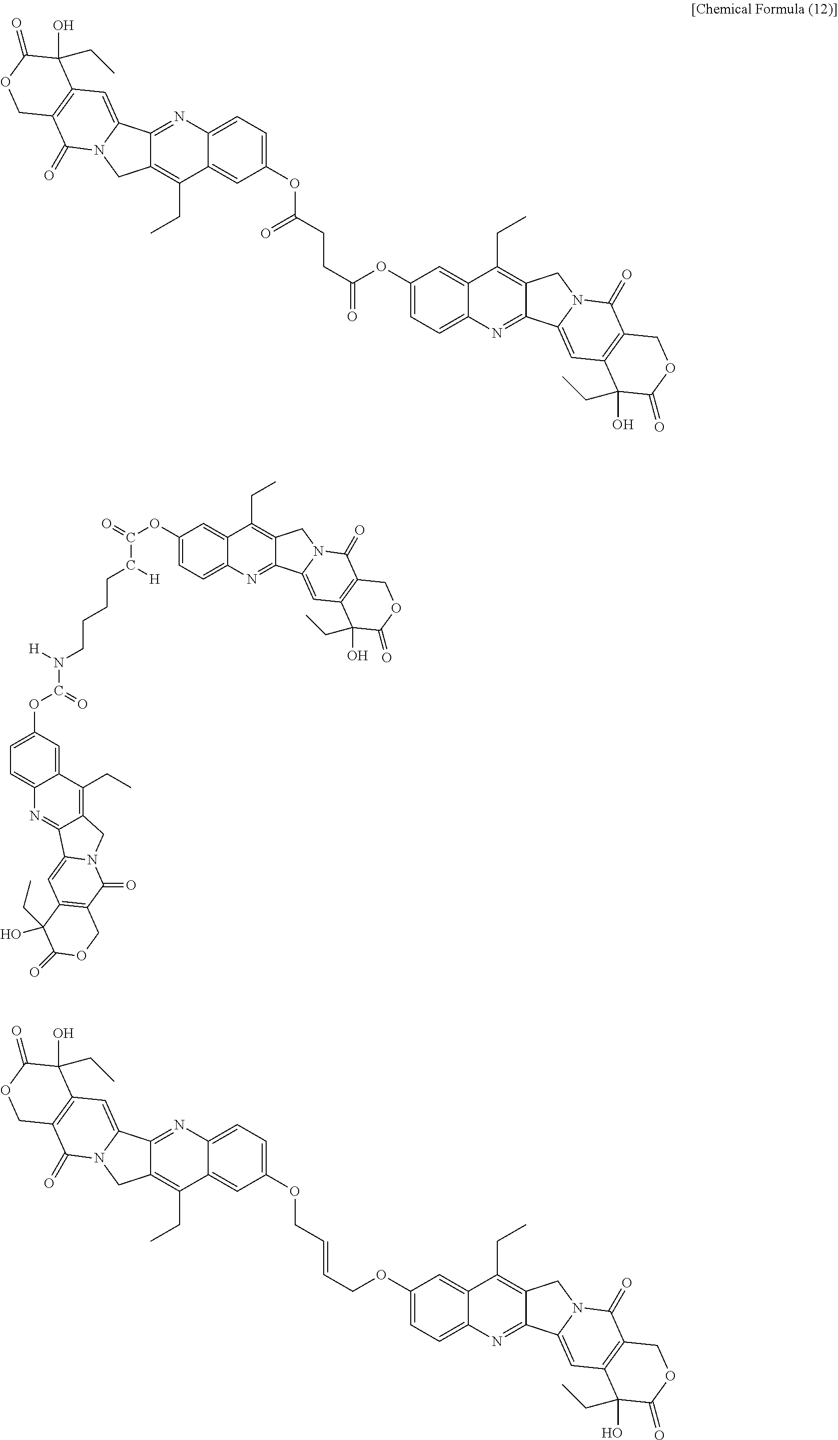 [SMILES: CCc1c2c(nc3ccc(OC(=O)CCC(=O)Oc4ccc5nc6c(c(CC)c5c4)Cn4c-6cc5c(c4=O)COC(=O)C5(O)CC)cc13)-c1cc3c(c(=O)n1C2)COC(=O)C3(O)CC ; CCc1c2c(nc3ccc(OC(=O)CCCCCNC(=O)Oc4ccc5nc6c(c(CC)c5c4)Cn4c-6cc5c(c4=O)COC(=O)C5(O)CC)cc13)-c1cc3c(c(=O)n1C2)COC(=O)C3(O)CC ; CCc1c2c(nc3ccc(OC/C=C/COc4ccc5nc6c(c(CC)c5c4)Cn4c-6cc5c(c4=O)COC(=O)C5(O)CC)cc13)-c1cc3c(c(=O)n1C2)COC(=O)C3(O)CC]

### Effect of the Invention

According to the present invention, pharmaceutical multimeric particles having small particle size and narrow particle size distribution can be obtained easily by using reprecitipation method established as a technique for producing superfine particles. The reprecitipation method can be applied to a drug having a structure in which monomer is hard to give particles, by converting the drug to a pharmaceutical multimer having a structure to which the reprecitipation method can be applied easily. This pharmaceutical multimer regenerates the original drug by hydrolysis in vivo, that is, practically works as pharmaceutical particle itself, thus the medicinal effect and safety can be ensured. According to the present invention, organic particles having sufficiently small particle size and narrow particle size distribution can be provided. Since the particles of the present invention can be produced by using the technique of the reprecitipation method as it is, the particles can be obtained conveniently and at low cost. In addition, according to the manufacturing method of the present invention, particles having sufficiently small particle size can be obtained in the combination of an organic compound in which the particle size becomes big with a traditional reprecitipation method and a poor solvent.

### BRIEF DESCRIPTION OF THE DRAWINGS

FIG. 1 shows the particle size of dimer of SN-38 and succinic acid.

FIG. 2 shows the particle size of dimer of SN-38 and tetramethylene diisocyanate.

FIG. 3 shows the anticancer activity of nanoparticles of SN-38 dimer against HepG2.

FIG. 4 shows the change in anticancer activity against HepG2 in aging of aqueous dispersion solution of SN-38 dimer nanoparticles

FIG. 5 shows the effect of linking mode used for producing SN-38 dimer on anticancer activity against HepG2.

### MODE FOR CARRYING OUT THE INVENTION

The present invention relates to preparing high quality pharmaceutical multimeric particles by multimerization of drug and application of reprecitipation method. Hereinafter, the present invention will be described in detail, and the basic manufacturing method of organic particles by reprecitipation method is disclosed in the aforementioned US2007041093, thus it can be referenced.

The particles manufactured in the present invention are multimers of a drug represented by the following general formula (1).

[Chemical Formula (13)]

Aa)ₖ (1)

In the general formula (1), A is a k-valent organic group, that is, a residue formed by removing k of substituent a from the drug represented by general formula (1). k is an integer of 2 or more, and a is a monovalent substituent. k of a may be the same or different each other.

The pharmaceutical multimer used in the present invention can be obtained by intermolecular dehydration condensation among a of plural drugs represented by the general formula (1). The bond formed by this dehydration condensation can be hydrolyzed in vivo by change of pH and action of enzyme to regenerate two original substituent a, that is, can be hydrolyzed to plural drugs represented by the general formula (1).

As the substituent represented by a, preferred are hydroxyl group, amino group, carboxyl group, thiol group, formyl group, hydrogen atom (provided that, limited to the hydrogen atom on secondary amine), and the like, and particularly preferred are hydroxyl group, amino group, carboxyl group and hydrogen atom (provided that, limited to the hydrogen atom on secondary amine). In addition, as the bond formed by condensation of certain two of a, preferred are —NHCO—, —COO— and —OCOO—, —COS—, and the like, and particularly preferred are —NHCO— and —COO—.

In the general formula (1), k is an integer of 2 or more, and although there is no upper limit, k is usually 5 or less, preferably 3 or less, particularly preferably 2, and specifically the structure represented by the following general formula (2) is preferred.

[Chemical Formula (14)]

a-A-b (2)

In the general formula (2), A is a divalent organic group, and a and b are each a substituent selected from hydroxyl group, amino group, carboxyl group, thiol group, hydrogen atom (provided that, limited to the hydrogen atom on secondary amine). The drug represented by the general formula (2) is converted to a pharmaceutical multimer having the structure represented by the following general formula (3) and microparticulated.

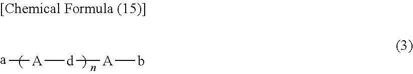

In the general formula (3), A, a and b are as defined in the general formula (2), n is an integer of 1 or more, and d is a divalent substituent wherein by the hydrolysis of d in vivo, the compound represented by the general formula (3) is converted to n+1 of the drug represented by the general formula (2).

d is a substituent obtained by intermolecular dehydration condensation of substituent a and b possessed by the drugs represented by the general formula (2), and examples of d are usually any one of —NHCO—, —COO—, —OCOO— and —COS—, and among them, —NHCO— and —COO— are preferred.

As n in the pharmaceutical multimer represented by the general formula (3), although there is no upper limit, n is usually 4 or less, preferably 2 or less, and particularly preferably 1.

In the present invention, the pharmaceutical multimer may be the one that drugs are linked each other via polyfunctional compound. For example, the drug represented by the general formula (4) is converted to a pharmaceutical multimer having a structure represented by the following general formula (5), and can be microparticulated.

[Chemical Formula (16)]

a-A (4)

Bd-A)ₘ (5)

In the general formula (4) and general formula (5), A is a monovalent organic group. In the general formula (4), a is a substituent selected from hydroxyl group, amino group, carboxyl group, thiol group and hydrogen atom (provided that, limited to the hydrogen atom on secondary amine). In the general formula (5), m is an integer of 2 or more, B is a m-valent substituent, and d is a divalent substituent wherein by hydrolysis of d in the living body, the compound represented by the general formula (5) is converted to the compound represented by B(-f)m wherein f is a substituent selected from hydroxyl group, amino group, carboxyl group and thiol group, and m of drug represented by the general formula (4).

d is a substituent obtained by dehydration condensation between substituent a possessed by the drug represented by the general formula (4) and substituent f possessed by the compound represented by the B(-f)m, and as d, usually exemplified is any one of —NHCO—, —CONH—, —COO—, —OCO—, —OCOO—, —COS— and —SCO—, among them, any one of —NHCO—, —CONH—, —COO— and —OCO— is preferred.

As m in the pharmaceutical multimer represented by the general formula (5), although there is no upper limit, m is usually 4 or less, preferably 3 or less, and particularly preferably 2.

The compound represented by the B(-f)m which is formed by in vivo hydrolysis of the pharmaceutical multimer represented by the general formula (5), is needed to be a safe compound for living body. Thus it is preferred to be a compound approved for administration to living body. Specifically, preferred is a compound having 2 or more groups selected from hydroxyl group, amino group, carboxyl group and thiol group, such as those described in Japanese Pharmaceutical Excipients Directory. Examples of such compound include, for example, succinic acid, fumaric acid, maleic acid, tartaric acid, malic acid, ascorbic acid, citric acid, lactic acid, amino acid, malonic acid, adipic acid, triethanolamine, trometamol, glycerin, and the like.

D in the general formula (I) represents an anticancer agent, for example, Camptothecin and derivatives thereof (e.g., 10-hydroxycamptothecin, 7-ethyl-10-hydroxycamptothecin (SN-38), 9-aminocamptothecin, etc.) are exemplified. 7-Ethyl-10-hydroxycamptothecin (SN-38) is preferred.

L in the general formula (I) represents a group selected from a group consisting of an optionally substituted alkyl, an optionally substituted cycloalkyl, an optionally substituted alkenyl, an optionally substituted alkynyl and an optionally substituted aryl. An optionally substituted alkyl and an optionally substituted alkenyl are preferred.

R in the general formula (I) represents a group selected from a group consisting of —C(═O)—, —C(═O)O—, —C(═O)NR1-, —C(═O)OC(═O)—, —C(═O)OC(═O)O—, —O—, —NHR1-, —S(═O)—, —SO2-, —SO(NR1)-, —SO2NR1, —P(═O)(OR1)O—, —P(═O) (NR)O— and alkylene, and —C(═O)—, —C(═O)NR1- and alkylene are preferred.

R1 represents a hydrogen, alkyl, an optionally substituted cycloalkyl or an optionally substituted aryl, and preferred is a hydrogen.

The “alkyl” indicates a saturated aliphatic hydrocarbon group, for example, a C1-C20 straight or branched group. Preferred examples of alkyl group include medium size alkyl having 1 to 10 carbon atoms, such as methyl, ethyl, propyl, 2-propyl, n-butyl, iso-butyl, tert-butyl, pentyl, and the like. More preferably, alkyl group is a lower alkyl having 1 to 4 carbon atoms, such as methyl, ethyl, propyl, 2-propyl, n-butyl, iso-butyl or tert-butyl. The alkyl group may be substituted or unsubstituted. When substituted, the substituent includes a halogen, hydroxyl, lower alkoxyl, and the like.

The “cycloalkyl” indicates a 3- to 8-membered monocyclic ring totally composed of carbon atoms, a 5-membered/6-membered or 6-membered/6-membered fused bicyclic ring totally composed of carbon atoms, or multifused ring (“fused” ring system means that each ring of the system shares carbon atom and adjacent pair with the other ring within the system) wherein one or more rings may contain 1 or more double bonds, but none of the rings has completely conjugated π-electron system. Examples of the cycloalkyl group include cyclopropane, cyclobutane, cyclopentane, cyclopentene, cyclohexane, cyclohexadiene, adamantane, cycloheptane, cycloheptatriene, and the like. The cycloalkyl group may be substituted or unsubstituted. When substituted, the substituent includes a lower alkyl, trihaloalkyl, halogen, hydroxy, lower alkoxyl, and the like.

The “alkenyl” indicates the above defined alkyl group having at least two carbon atoms and at least one carbon-carbon double bonds. Representative examples include ethenyl, 1-propenyl, 2-propenyl, 1-, 2-, 3-butenyl, and the like, and ethenyl is preferred.

The “alkynyl” indicates the above defined alkyl group having at least two carbon atoms and at least one carbon-carbon triple bonds. Representative examples include ethynyl, 1-propynyl, 2-propynyl, 1-, 2-, 3-butynyl and the like, but are not limited to these.

The “aryl” indicates a group having at least one aromatic ring, namely, a group having a conjugated π-electron system, such as cyclic aryl totally composed of carbon atoms, heteroaryl and biaryl. In some cases, the above-mentioned aryl group may be substituted with 1 or more groups selected independently from the group consisting of halogen, trihalomethyl, hydroxy, SR, nitro, cyano, alkoxyl and alkyl.

Examples of the compound represented by the general formula (I) include the followings, but are not limited to these.

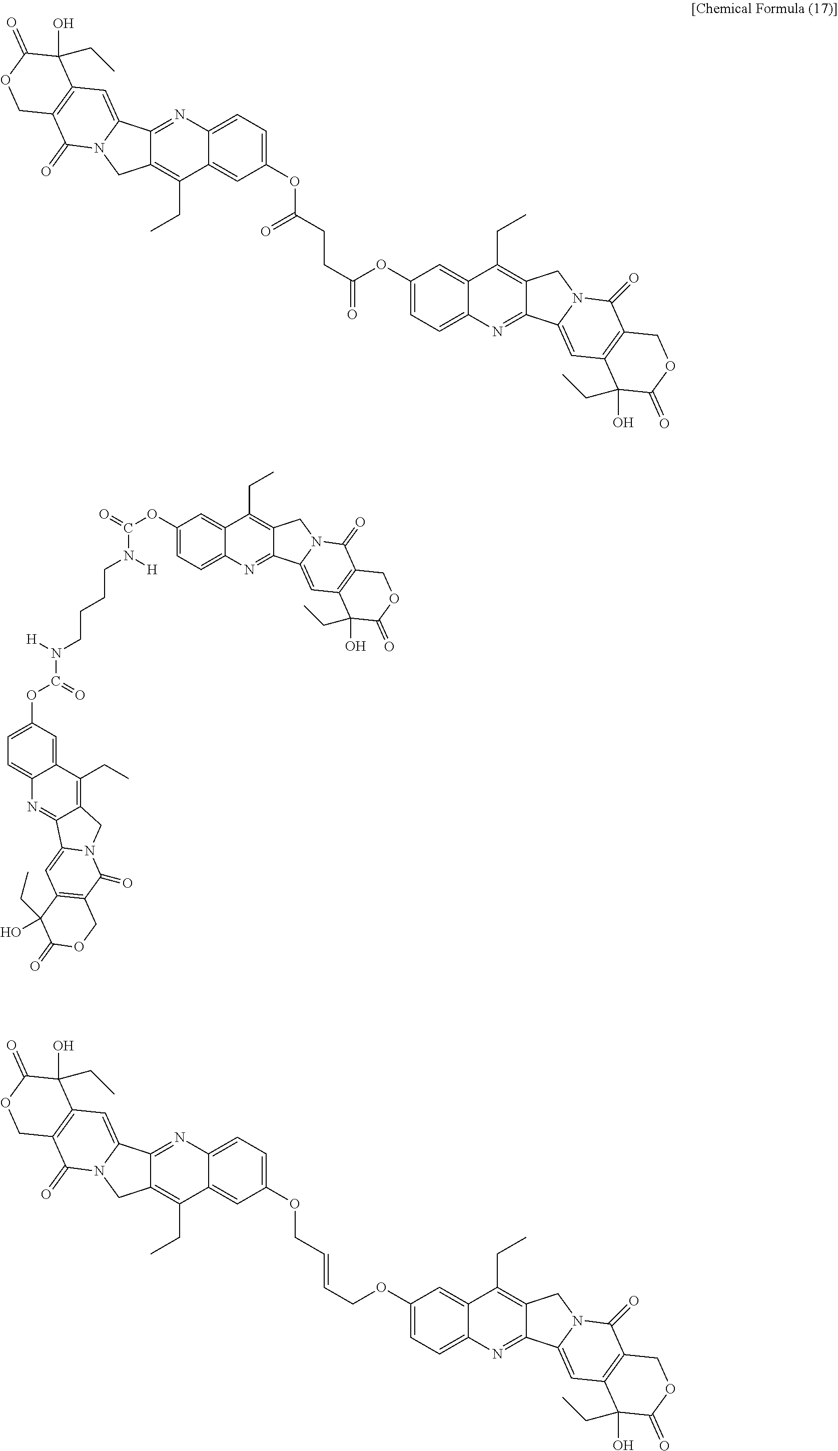

The drug represented by the general formula (1), general formula (2), general formula (3), general formula (4) and general formula (5) is preferred to be a poorly water-soluble drug having solubility of 1.0 g/L or less in 25° C. water, further 0.1 g/L or less, particularly 0.01 g/L or less, from the characteristics of reprecipitation method, namely, depositing by pouring a solution into water. In addition, preferable example of the kind of drugs includes an anti-inflammatory agent, a bronchodilator, an antibiotic, an antiviral agent, an anti-HIV drug, an immunosuppressive agent, an immune-stimulating agent, an anesthetic drug, an antimycotic agent, an antioxidant, an antidiabetic drug, hormone, vitamin, an antiepileptic drug, an anticancer agent, a muscle relaxant, a stimulating agent, an antitussive drug, a labor pain controlling agent, an anti-alcoholism drug, an antismoking agent, a hypotensive agent, an instillation, and the like. In addition, in the present invention, “drug” means the pharmaceutical agent prescribed in the Japanese Pharmacopeia and/or the Drugs in Japan Ethical Drugs.

Hereinafter, the combination of the drug represented by the general formula (1), general formula (2) or general formula (4) and the pharmaceutical multimer thereof is exemplified, but the present invention is not limited to these compounds and combinations.

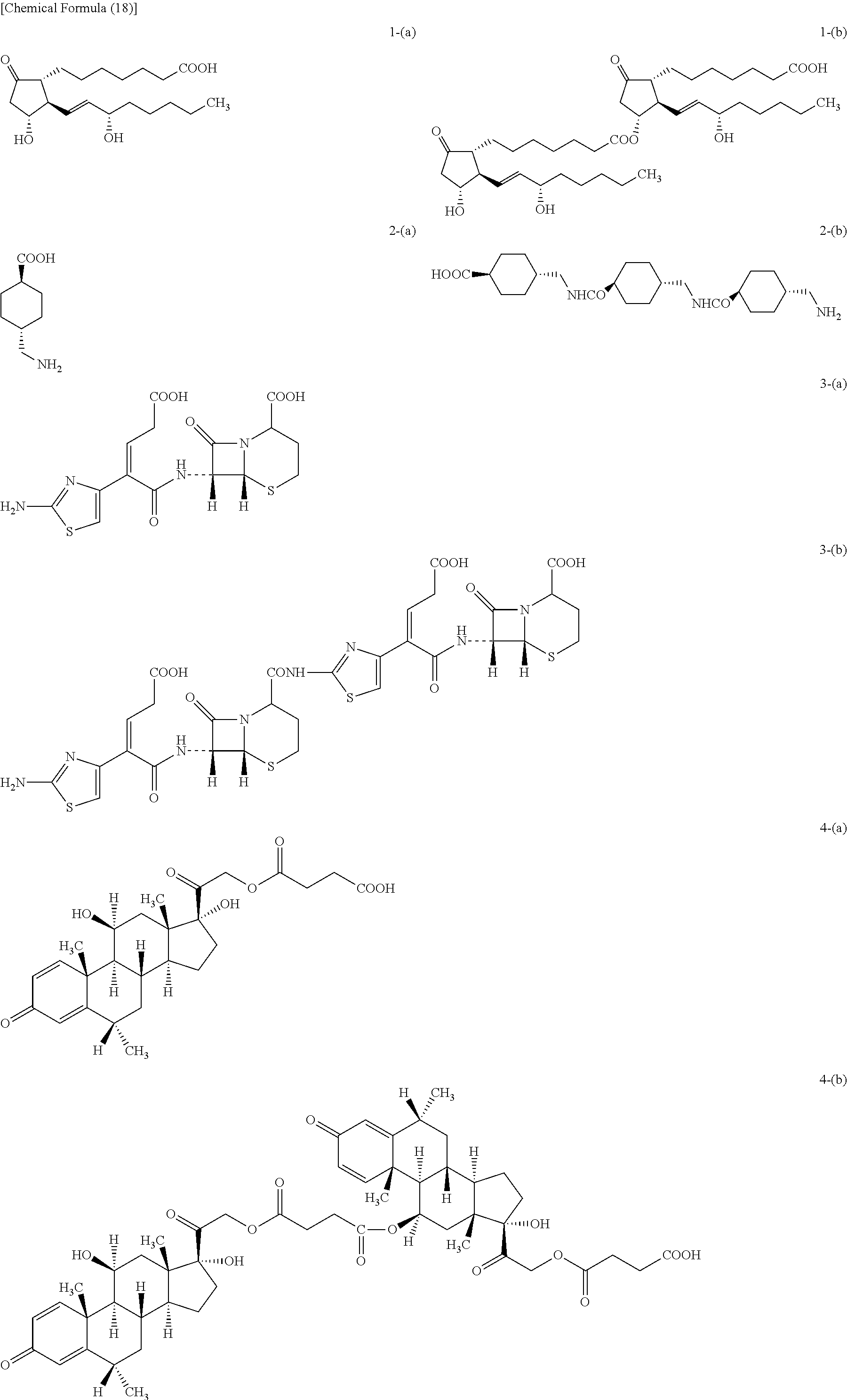

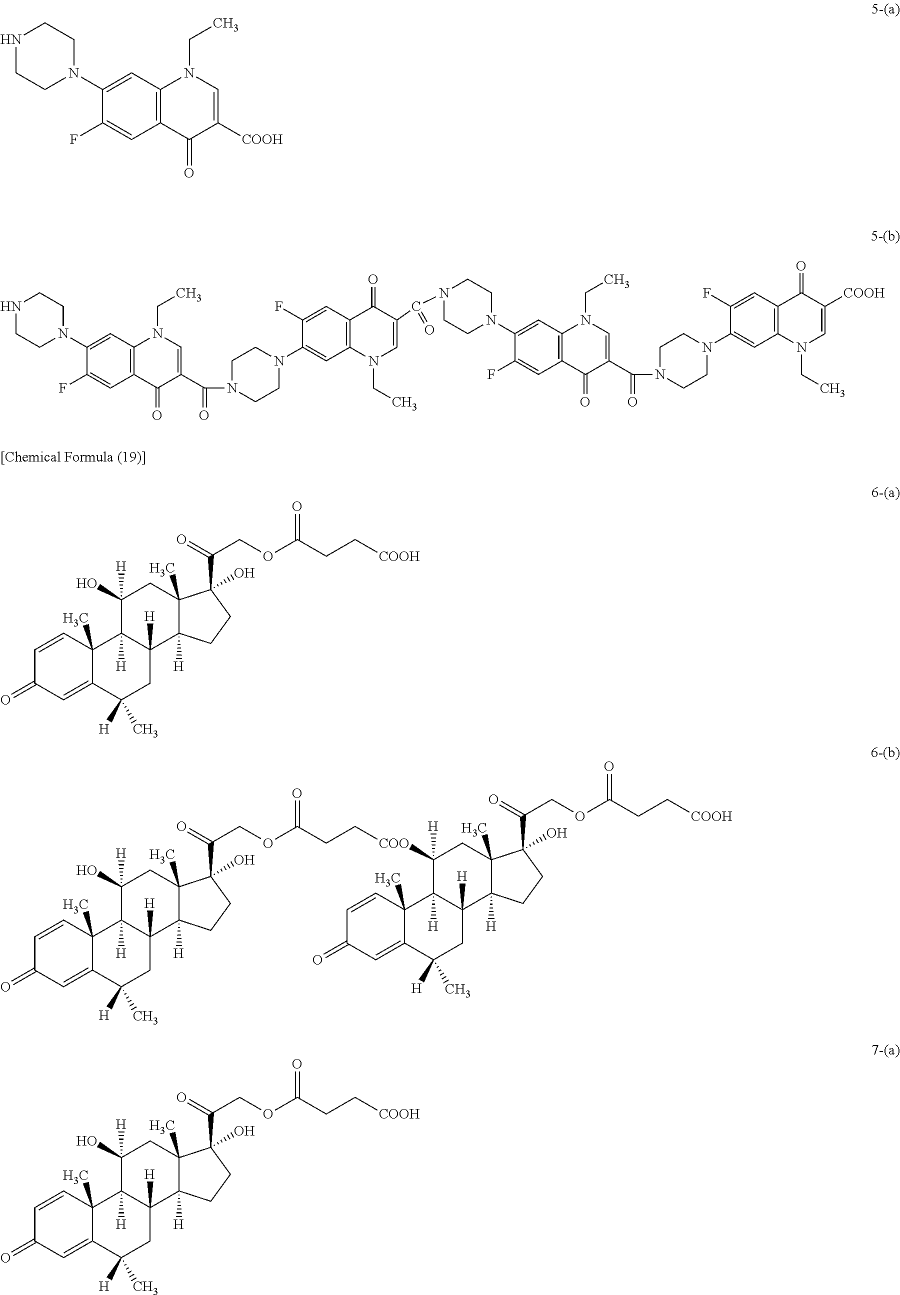

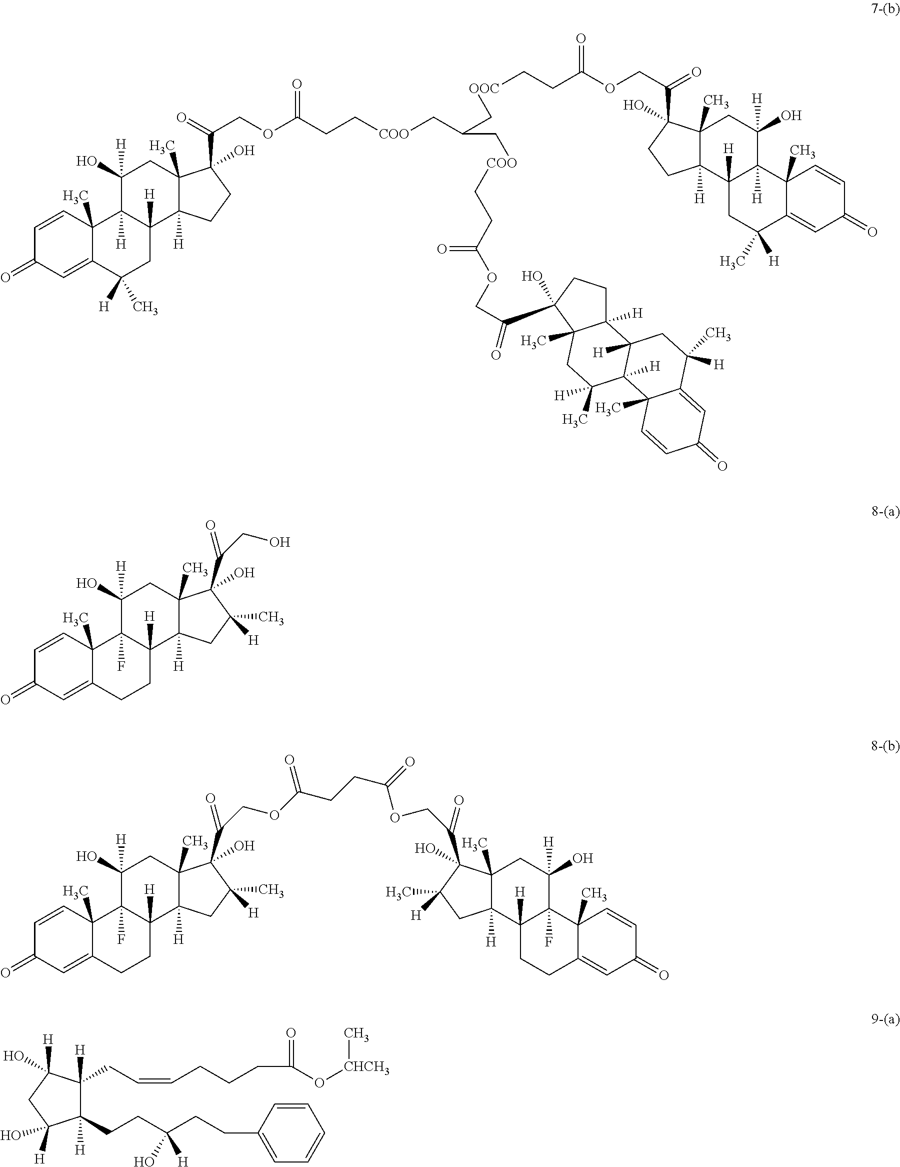

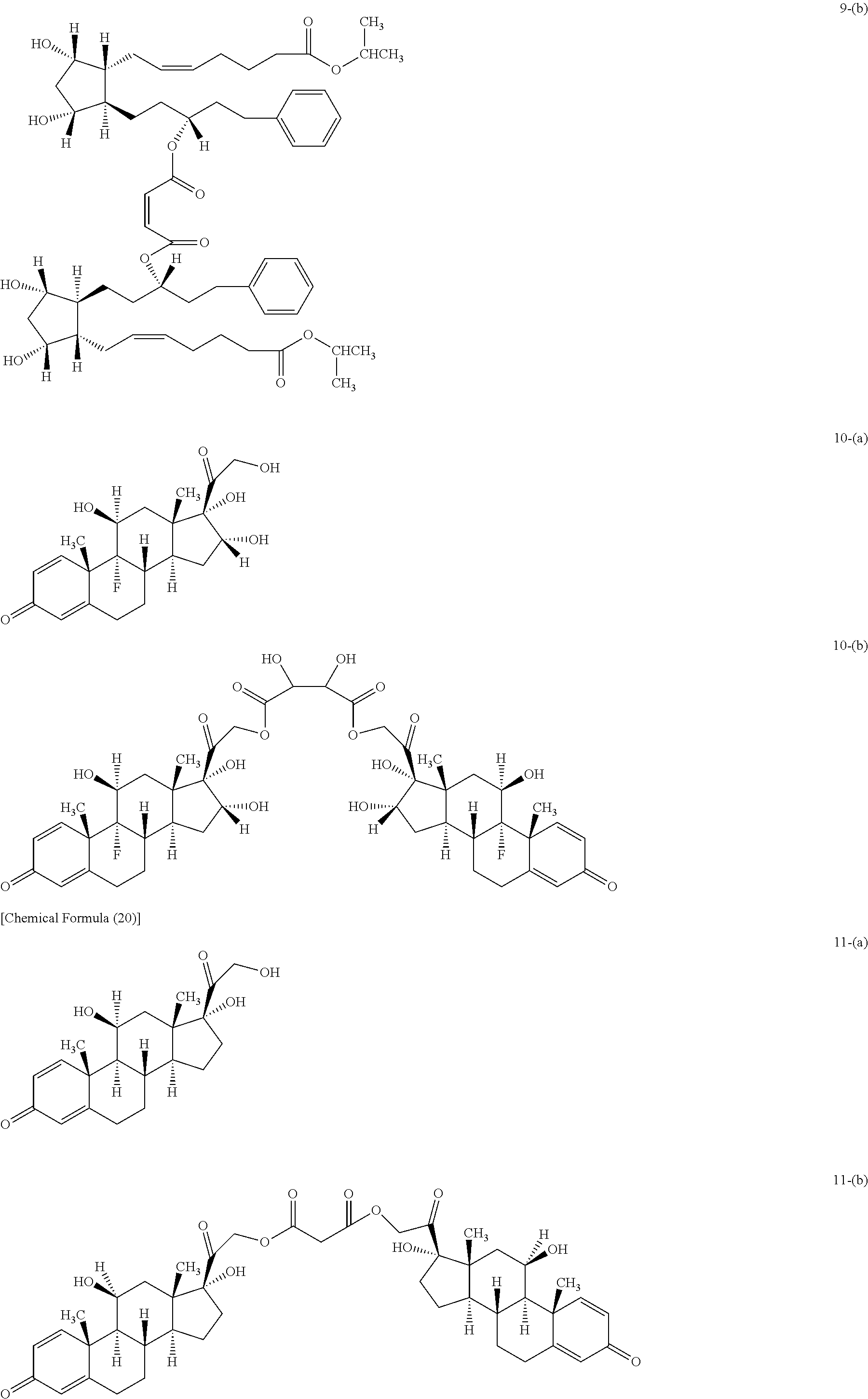

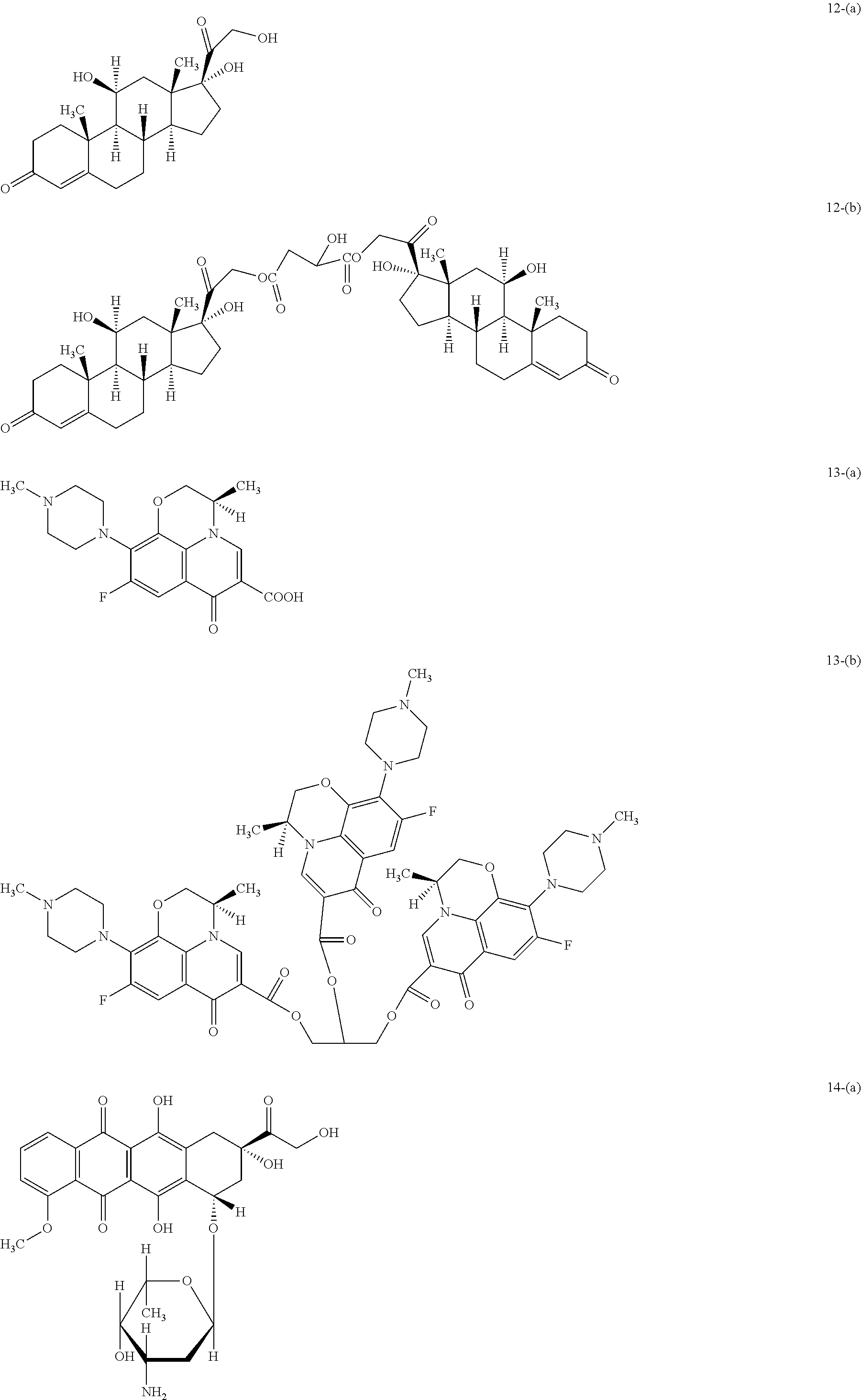

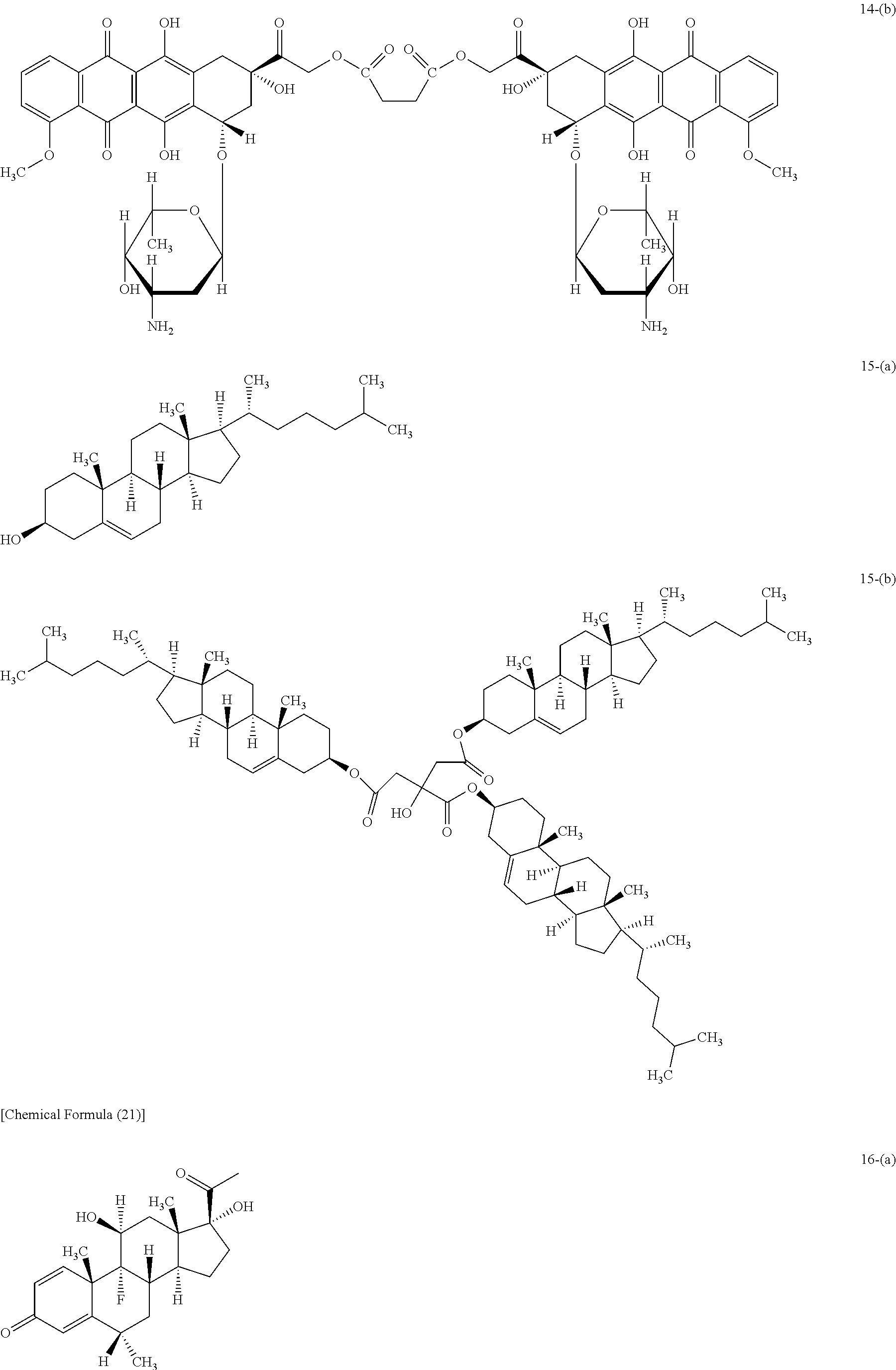

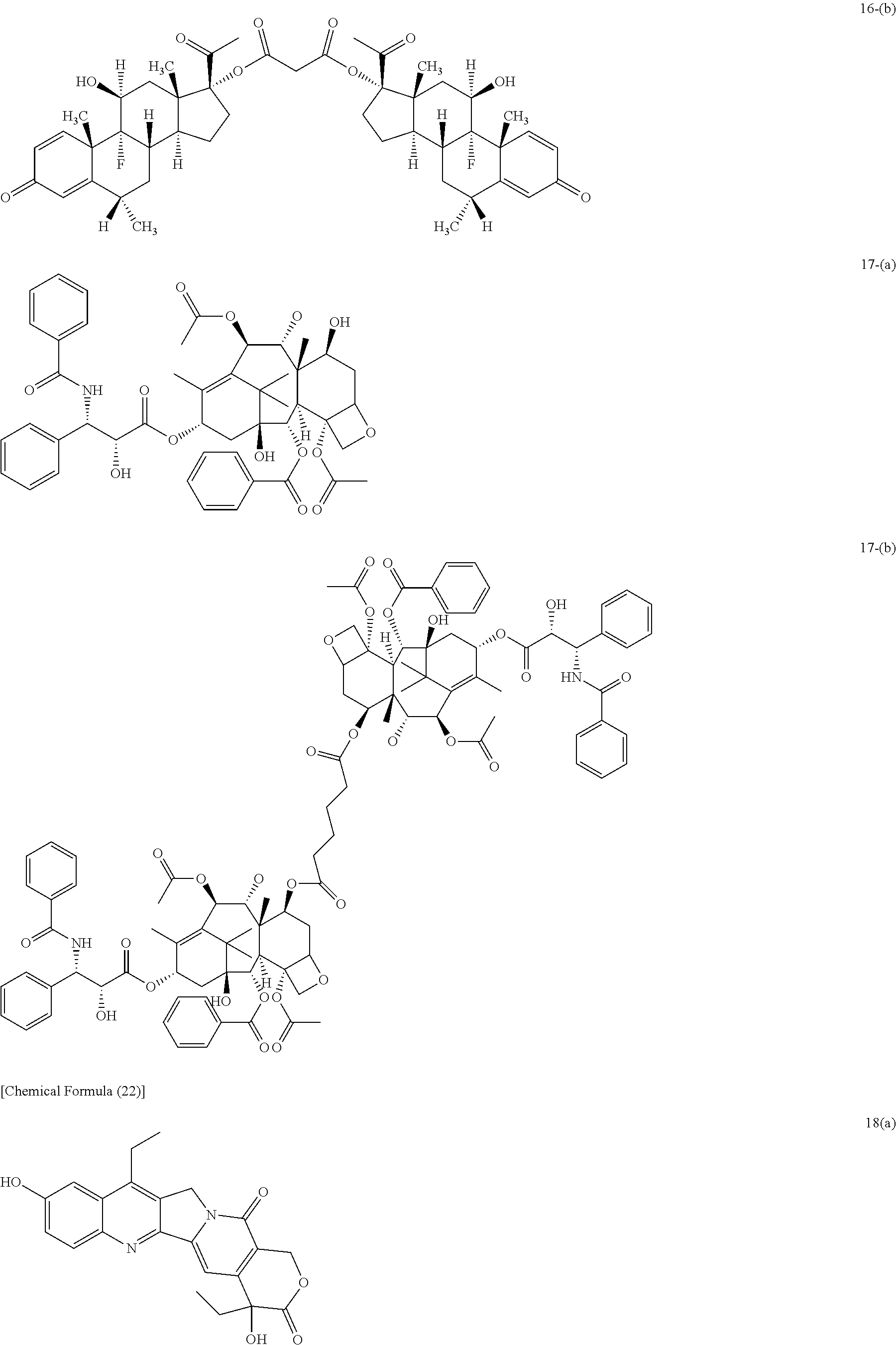

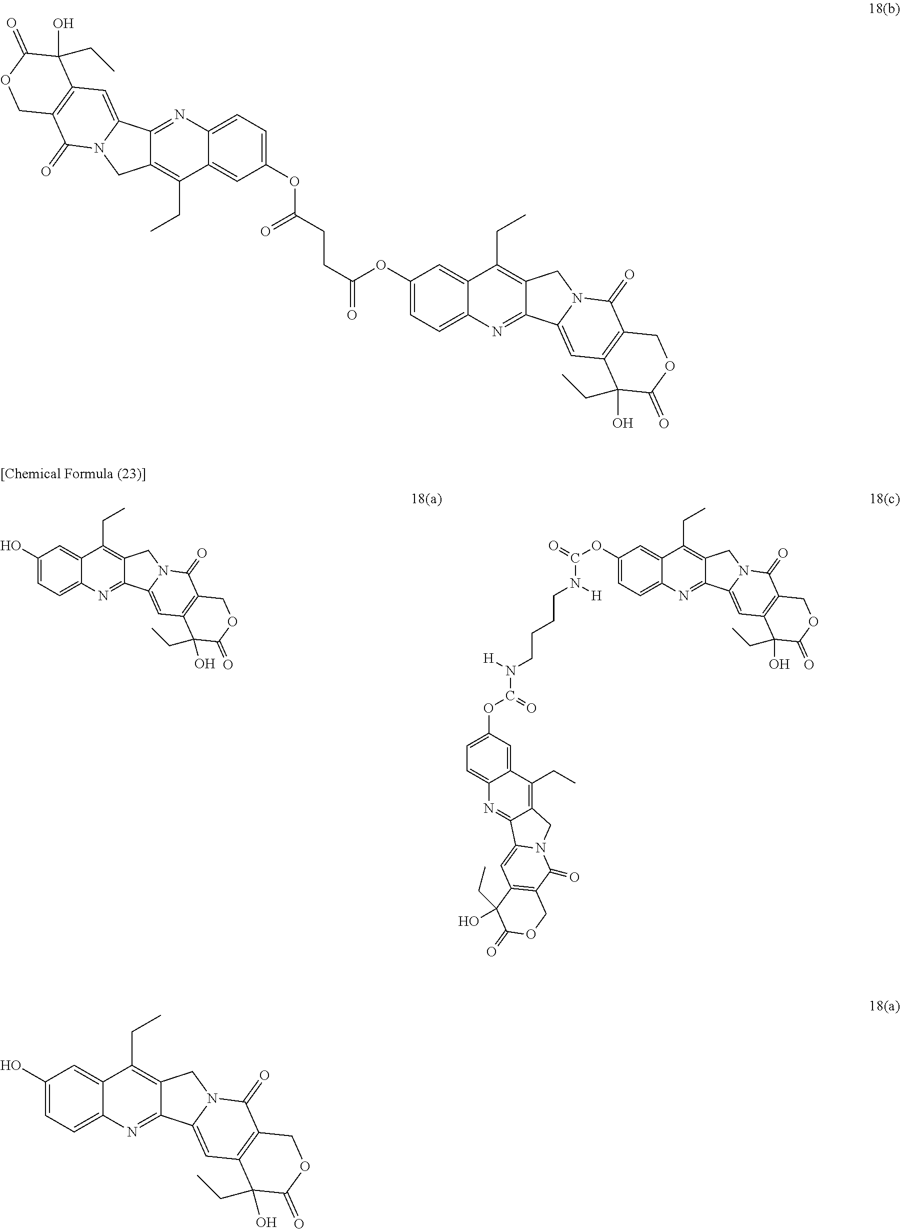

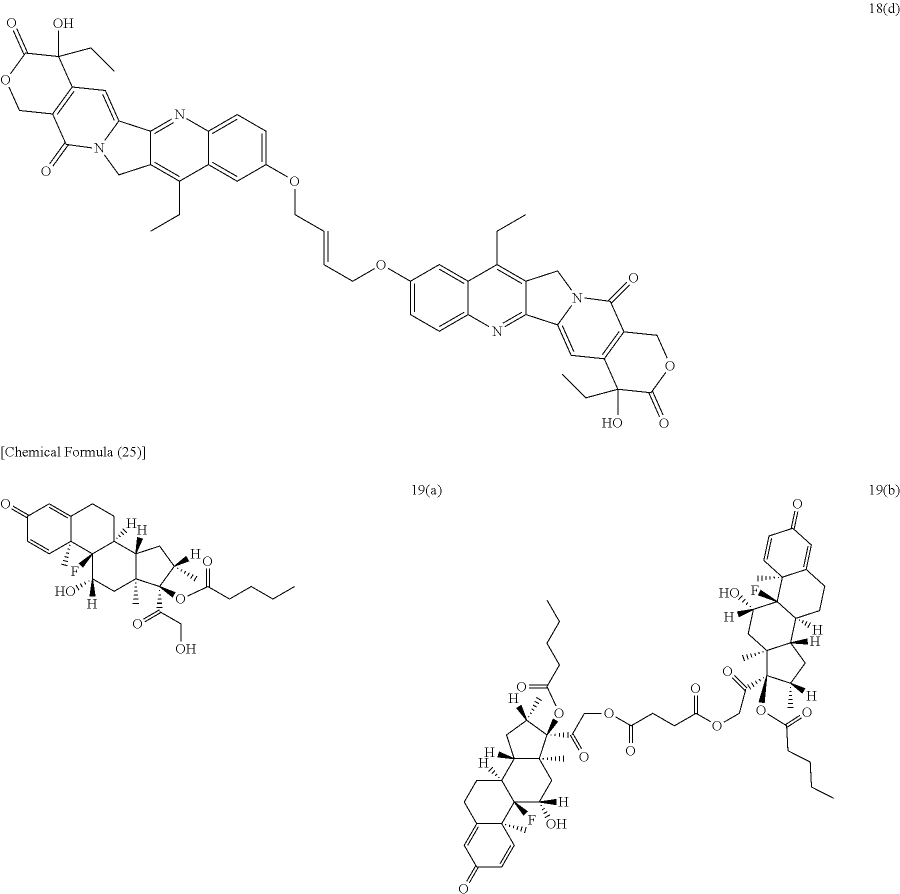

The pharmaceutical multimer of the present invention can be produced with a known method by a person skilled in the art. For example, it can be produced by subjecting plural drugs represented by the general formula (1) to intermolecular dehydration condensation among a.

The pharmaceutical multimer having the structure represented by the general formula (5) can be produced by reacting the drug represented by the general formula (4) with a polyfunctional compound.

As the polyfunctional compound, preferred is a compound having 2 or more substituents selected from hydroxyl group, amino group, carboxyl group, thiol group, halogencarbonyl group and isocyanate group. Example of halogencarbonyl group includes chlorocarbonyl group, bromocarbonyl group, etc., and chlorocarbonyl group is preferred.

The pharmaceutical multimer represented by the general formula (I) can be produced, for example, based on the method described in US2007041093.

In producing the pharmaceutical multimeric particles of the present invention, first, pharmaceutical multimer is dissolved in an organic solvent which is a good solvent. As long as an organic solvent can dissolve the pharmaceutical multimer and is miscible with water, it can be appropriately selected as the organic solvent. As specific organic solvents, depending on the compound to be dissolved, exemplified are acetone, tetrahydrofuran, dioxane, acetonitrile, methanol, ethanol, propanol, N-methylpyrrolidone, dimethyl sulfoxide, and the like, and these may be used alone or with mixture. As organic solvent, acetone, ethanol and dimethyl sulfoxide are preferred from a viewpoint of solubility and safety.

Because concentration of pharmaceutical multimer dissolved in an organic solvent is a major factor affecting the particle size of particles to be formed, the concentration is appropriately changed depending on the desired particle size. Solution concentration is usually adjusted to around 0.1 to 15% by mass, and when the molecular weight is high, a solution of about 0.5% by mass is preferably prepared.

When a solution of pharmaceutical multimer in an organic solvent (hereinafter, simply referred to as organic solvent solution) is poured into water which is a poor solvent, pharmaceutical multimeric particles are formed in a condition dispersed in water. In the present invention, the solubility of pharmaceutical multimer in water becomes lower than that of original drug by the presence of plural organic group A in the pharmaceutical multimer. As a result, the pharmaceutical multimer crystallizes more rapidly in water than the original drug, providing particles having smaller particle size and particle size distribution.

The amount of water wherein an organic solvent solution is poured into is usually more than 10 times based on the volume of pharmaceutical multimer. Although there is no upper limit, when water is too much, the concentration of the pharmaceutical multimeric particles becomes low and condensation is needed, thus usually the amount of water is not more than 100 times.

Since when the temperature of water wherein an organic solvent solution is poured into is changed, the particle size of the particles formed is also changed, the particle size of the pharmaceutical multimeric particles can be controlled by keeping the water at a given temperature. For example, when low temperature water is used, the particle size of the pharmaceutical multimeric particles tends to become large. Depending on the desired particle size, temperature is controlled usually within a range of −20° C. to 60° C.

Pouring of organic solvent solution is preferred to carry out quickly in a short time into water under stirring condition in order to prevent variation of particle size. By continuing stirring for a while after pouring, an aqueous dispersion can be obtained wherein the crystallized or microparticulated pharmaceutical multimers are dispersed uniformly in water. The obtained pharmaceutical multimeric particles can be used directly with the state dispersed in water as an aqueous suspension agent, and further can be isolated as powder of fine particles by carrying out a solid-liquid separation operation such as filtration.

When used as an aqueous suspension agent, although the aqueous dispersion can be used directly depending on the intended use and the kind of good solvent used, the organic solvent added as good solvent is generally removed so as to improve the safety as a drug. The method for removing the organic solvent is not limited, and it can be removed from the aqueous dispersion by a known method. The organic solvent can be removed easily by distilling away under reduced pressure (or ordinary pressure), and can also be removed by using dialysis.

By conducting the above-mentioned operation, pharmaceutical multimeric particles having very small particle size and narrow particle size distribution can be obtained, that is, it is possible to produce pharmaceutical multimeric particles having narrow particle size distribution and large particle size by controlling the water temperature, kind of good solvent, the amount added, etc., and usually pharmaceutical multimeric particles having mean particle size of about 50 nm to 10000 nm can be produced by altering the production condition.

Pharmaceutical multimers are hydrolyzed in vivo to the form of original drug by the change in pH during transit through the living body and action of enzyme such as esterase. Therefore, the medicinal effect and safety of the pharmaceutical multimeric particles of the present invention can be ensured since they serve in vivo as the drug itself before multimerizing.

Hereinbefore, the present invention is described in reference to the most practical and preferred embodiment at this time, but the present invention is not limited to the embodiment disclosed in the specification of the present application, and appropriately changes may be made without departing from the scope or ideas of the invention read from the claims and whole description of specification, thus the pharmaceutical multimeric particles accompanying such changes and the production method thereof must be understood to be included in the technical scope of the present invention.

The present invention relates to the production of high-quality organic particles having small particle size by a reprecipitation method wherein a compound to become a crystal nucleating agent is used together. Hereinafter, the present invention will be described in detail, and the basic production method of the organic particles by a reprecipitation method is disclosed in the above-mentioned US2007041093, thus it can be used as a reference.

The organic particles manufactured in the present invention are particles of a compound represented by the following general formula (6).

[Chemical Formula (26)]

A′a′)_{k′} (6)

In the general formula (6), A′ is a k-valent organic group having an optional carbon skeleton, k′ is an integer of 1 or more, and a′ is a hydrogen atom or a monovalent substituent. The organic group A′ is selected to be a main skeleton of the compound represented by the general formula (6). Specifically, the number of atoms in the general formula (6) is in relation of (number of atoms possessed by the patial structure A′>number of atoms possessed by the other part). The compound represented by the general formula (6) is preferably selected in such a way that A′ includes an aromatic and/or non-aromatic cyclic organic group and a′ doesn't include an aromatic and/or non-aromatic cyclic organic group, and examples of the compound represented by the general formula (6) include an aromatic polycyclic compound such as phthalocyanine, quinacridone, perylene and indanthrene, and a non-aromatic polycyclic compound such as steroidal drugs.

Preferable substituent as a″ is a monovalent group wherein the atom number of main chain except hydrogen atom is 1 to 3, or a hydrogen atom, and for example, methyl group, propyl group, hydroxyl group, methoxy group, acetyl group, methoxycarbonyl group, methylcarbamoyl group, carboxyl group, amino group, sulfo group, halogen atom, cyano group, nitro group, and the like are exemplified. Among them, carboxyl group, hydroxyl group and amino group are preferred, and particularly preferred is carboxyl group, hydroxyl group, amino group and halogen atom.

In addition, in this specification, the “atom number of main chain except hydrogen atom” means the maximum number of atoms (except hydrogen atom) counted tandemly from the atom bonded to A′, and for example, propyl group is 3, isopropyl group is 2, acetyl group is 2, carboxyl group is 2, sulfo group is 2, amino group is 1 and halogen atom is 1.

In the general formula (6), k′ is an integer of 1 or more, and there is no upper limit, usually k′ is 5 or less, preferably 3 or less, particularly preferably 2, and specifically, preferred is the structure represented by the following general formula (7).

[Chemical Formula (27)]

′a-A′-b′ (7)

In the general formula (7), A′ is a divalent organic group having an optional carbon skeleton, and a′ and b′ are each independently a hydrogen atom or a monovalent substituent. The organic group A′ is selected to be a main skeleton of the compound represented by the general formula (7). Specifically, the atom number of partial structure A′, a′ and b′ is in relation of A′>(a′+b′). The compound represented by the general formula (7) is preferably selected in such a way that A′ includes an aromatic and/or non-aromatic cyclic organic group and a′ and b′ don't include an aromatic and/or non-aromatic cyclic organic group, and examples of the compound represented by the general formula (7) include an aromatic polycyclic compound such as phthalocyanine, quinacridone, perylene and indanthrene, and a non-aromatic polycyclic compound such as steroidal drugs.

Preferred substituents as a′ and b′ include the same groups as those exemplified with respect to a′ of general formula (6), and preferable groups are also the same.

The compound represented by the general formula (6) or general formula (7) is dissolved in organic solvent together with a crystal nucleating agent having plural partial structure A possessed by the compound represented by the general formula (6) or general formula (7). For example, the compound represented by the general formula (7) is dissolved in organic solvent together with the compound represented by the following general formula (8).

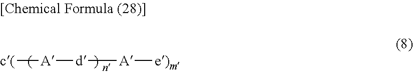

In general formula (8), A is an organic group having the same skeleton as A′ in the general formula (7). By having plural organic group A, the solubility of the compound represented by the general formula (8) in a poor solvent becomes lower than that of the compound represented by the general formula (7). As the result, the compound represented by the general formula (8) deposits in the poor solvent ahead of the compound represented by the general formula (7), and becomes a crystal nucleating agent for the compound represented by the general formula (7).

In the general formula (8), d′ is a divalent linking group or simply a bond. The “d′ is simply a bond” means the state that a given organic group A′ and the adjacent organic group A′ are bonded directly without intervening substituent. As the divalent linking group, preferred is a substituent group wherein the atom number of main chain except hydrogen atom is 1 to 3, and the examples include —(CH2)ₓ- (provided that x represents an integer of 1 to 3), —COO—, —CONH—, —COOCO—, —CON(R1)- (provided that R1 represents a hydrogen atom or a hydrocarbon group), —OCO—, —CH2OCO—, —O—, —S—, and the like. More preferably d′ is any one of simply a bond, —OCO—, —COO—, —O—, —CONH— and —COOCO—.

In the general formula (8), e′ is a hydrogen atom or a monovalent substituent. As e′, the same groups as a′ in the general formula (6) are exemplified. As e′, particularly preferred is a monovalent substituent wherein the atom number of main chain except hydrogen atom is 1 to 3 or a hydrogen atom.

In the general formula (8), n′ is an integer of 1 or more, and m′ is an integer of 1 or more. m′ of e′, n′, and m′×n′ of d′ may be each the same or different, and preferably m′ of n′ and m′×n′ of d′ are each the same number, and the same structure. n′ is preferably 1 to 10, more preferably 1 or 2, and further preferably 1. In addition, m′ is preferably 1 to 4, and further preferably 1. Furthermore, the compound represented by the general formula (8) may be a mixture of plural kinds of compound having the structure represented by general formula (8).

In the general formula (8), c′ is a hydrogen atom or a m′-valent substituent. As c′, in case that c′ is a monovalent substituent, the same groups as a′ in the general formula (6) are exemplified, and in case that c′ is a divalent substituent, the same groups as d′ can be exemplified. In addition, examples of di- or more-valent substituent include residues of polyfunctional organic compound such as residues obtained by removing the hydrogen atom on hydroxyl group of polyhydric alcohols such as ethylene glycol, diethylene glycol, propylene glycol, glycerol, trimethylolpropane, diglycerin, etc.; residues obtained by removing hydroxyl group bonded to carbonyl of polycarboxylic acids such as oxalic acid, malonic acid, succinic acid, adipic acid, maleic acid, phthalic acid, 1,3,5-benzenetricarboxylic acid, etc., or removing the hydrogen atom on said hydroxyl group; residues obtained by removing hydrogen atom on amino group from polyamines such as ethylenediamine, phenylenediamine, 1,3,5-triaminobenzene, etc.; residues obtained by removing halogen atom from compound having plural halogen atoms such as dichlorobenzene, tribromobenzene, etc.; residues of compound having plural kinds of functional group in the molecule such as salicylic acid, lactic acid, ethanolamine, amino acids, etc.; and the like.

Preferable example of the compound represented by the general formula (8) includes a polyfunctional organic compound having the above-exemplified residue to which plural of the “reactive substituent a′ possessed by the compound represented by the general formula (7)” or “reactive substituent a″ converted from substituent a′ possessed by the compound represented by the general formula (7)” are linked by reacting the “reactive substituent a′ possessed by the compound represented by the general formula (7)” or “reactive substituent a″ converted from substituent a′ possessed by the compound represented by the general formula (7)” with said polyfunctional organic compound.

Further, the compound represented by the general formula (8) may preferably be the compound represented by the following general formula (9).

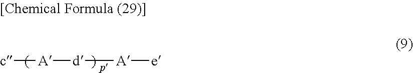

In the general formula (9), A′, d′ and e′ are each as defined in the general formula (8). Provided that p′ of d′ are all the same structure.

In the general formula (9), c″ is a hydrogen atom or a monovalent substituent. As c″, the same groups as a′ in general formula (6) are exemplified. As e′, particularly preferred is a monovalent substituent wherein atom number of main chain except hydrogen atom is 1 to 3, or a hydrogen atom.

In the general formula (9), p′ is an integer of 1 or more. The compound represented by the general formula (9) may be a mixture of plural compounds having different p′ value. Preferably, from the viewpoint of solubility, p′ is preferred to be 1 to 10, more preferably 1 or 2, and further preferably 1.

In the general formula (9), the atom numbers of A′, c″ and e′ which are partial structures are selected preferably to be in relation of (p′+1)A′>(c″+e″+p′d′).

As the compound represented by the general formula (9), exemplified are, for example, multimers of the compound represented by the general formula (7) which is obtained by reacting to condense intermolecularly the reactive substituents a′ and b′ possessed by the compound represented by the general formula (7); and those that plural organic group A′ are linked, which is obtained by converting a′ and/or b′ of the compound represented by the general formula (7) to reactive group a″ and/or b″, followed by reacting a′ or a″ with b′ or b″ intermolecularly, and the like.

The following represents a combination of the compound represented by the general formula (6) or general formula (7) and a crystal nucleating agent to be used together in the production of particles thereof, but the present invention is not limited to these compounds and combinations.

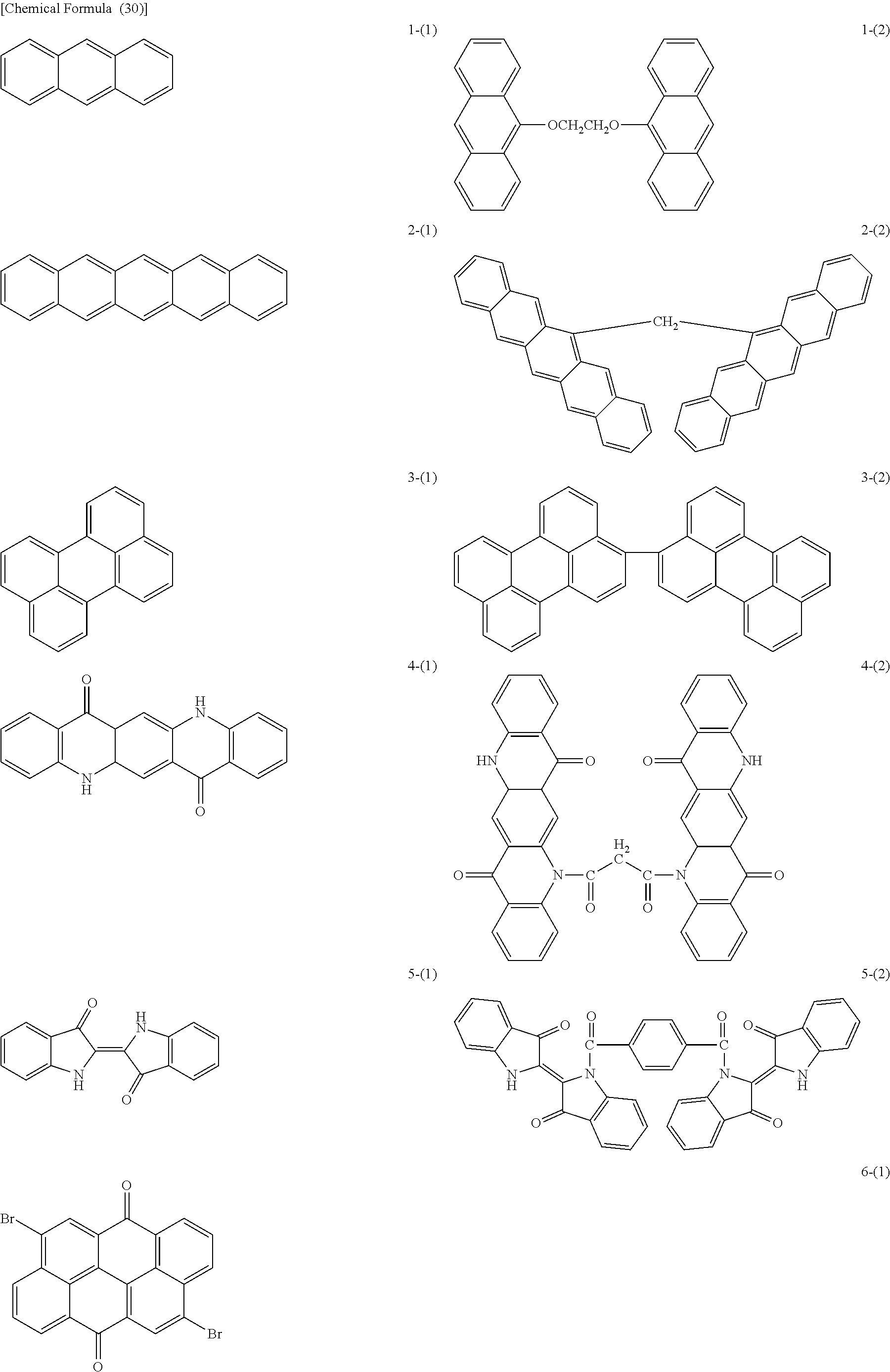

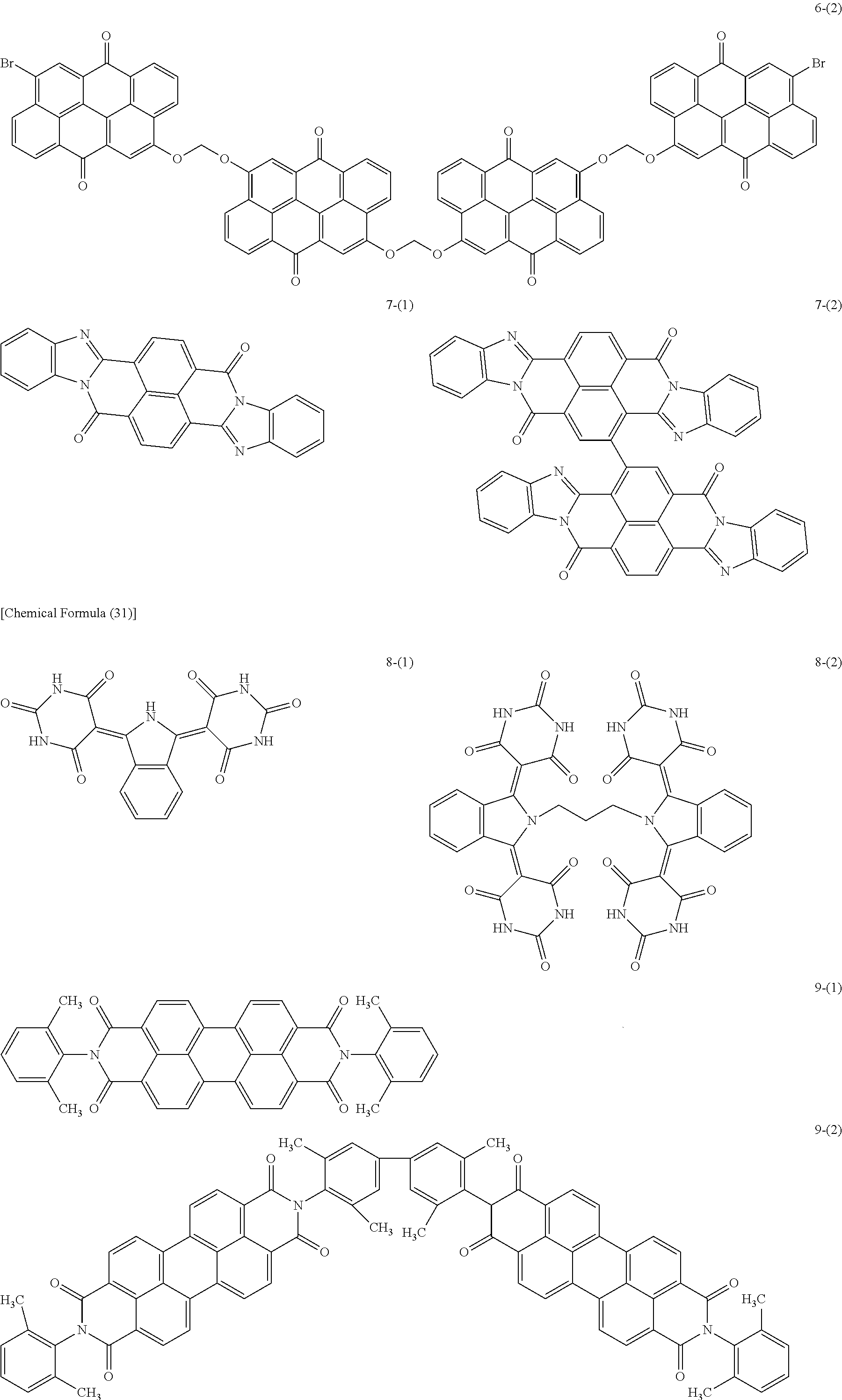

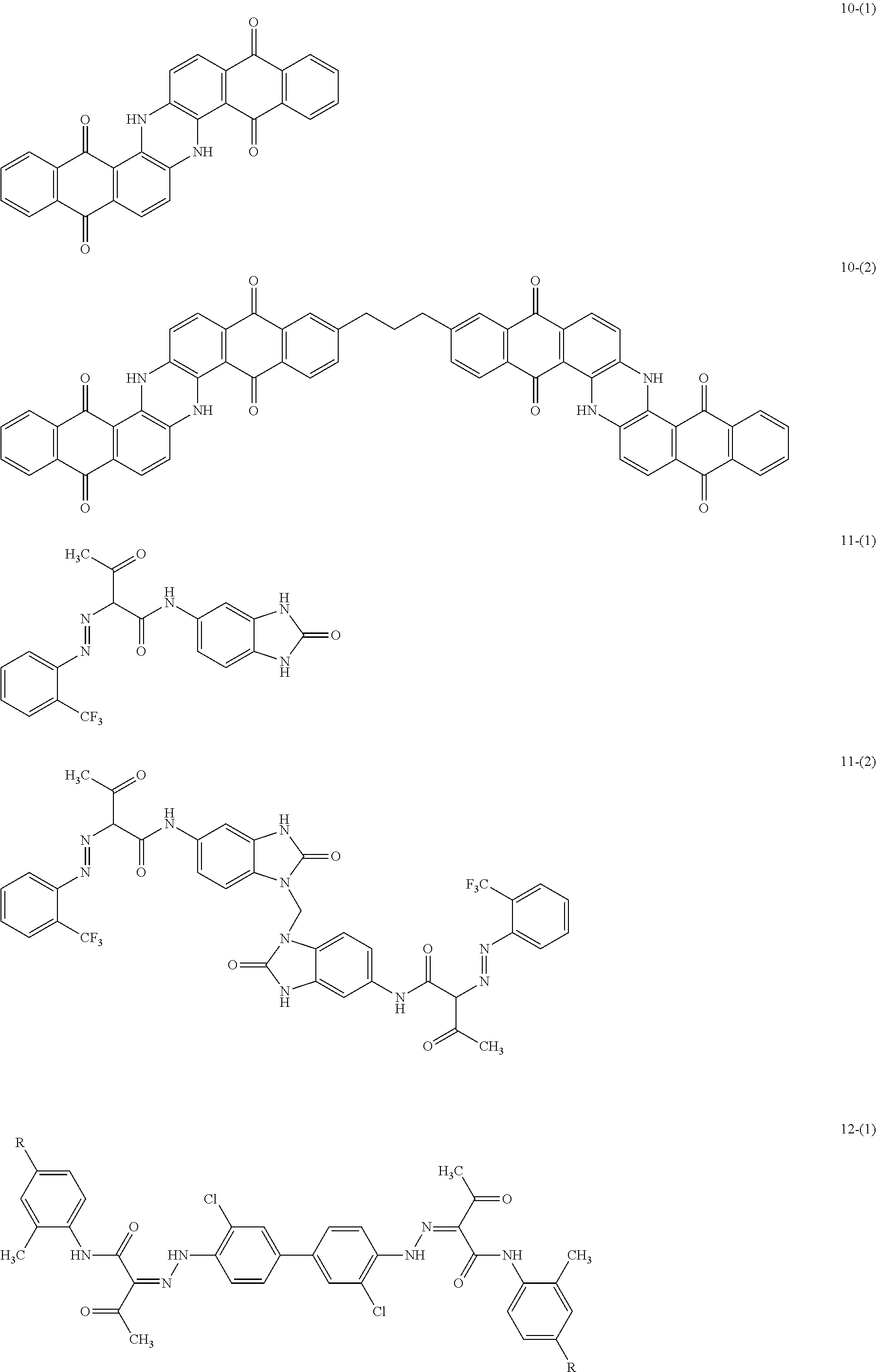

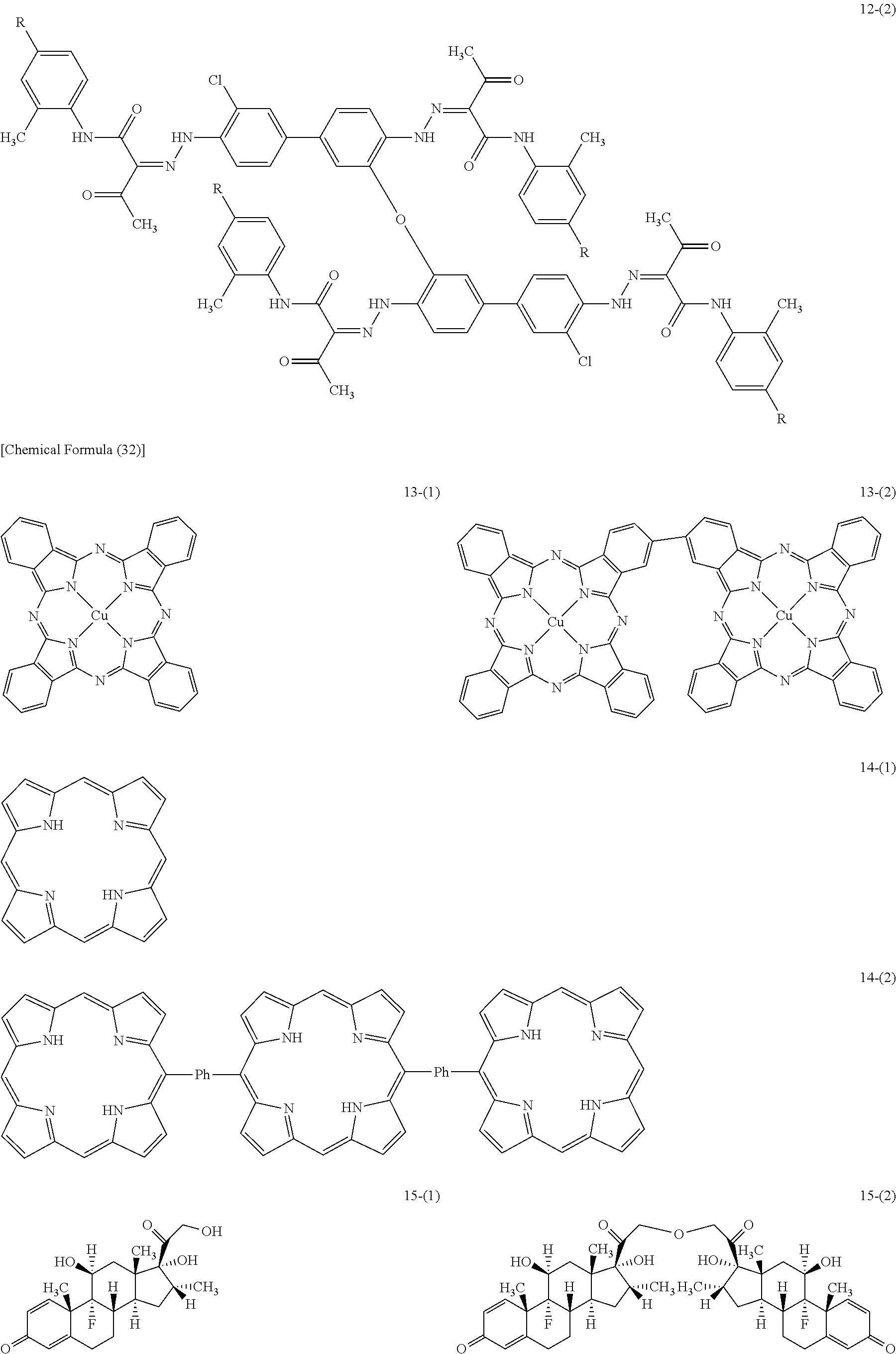

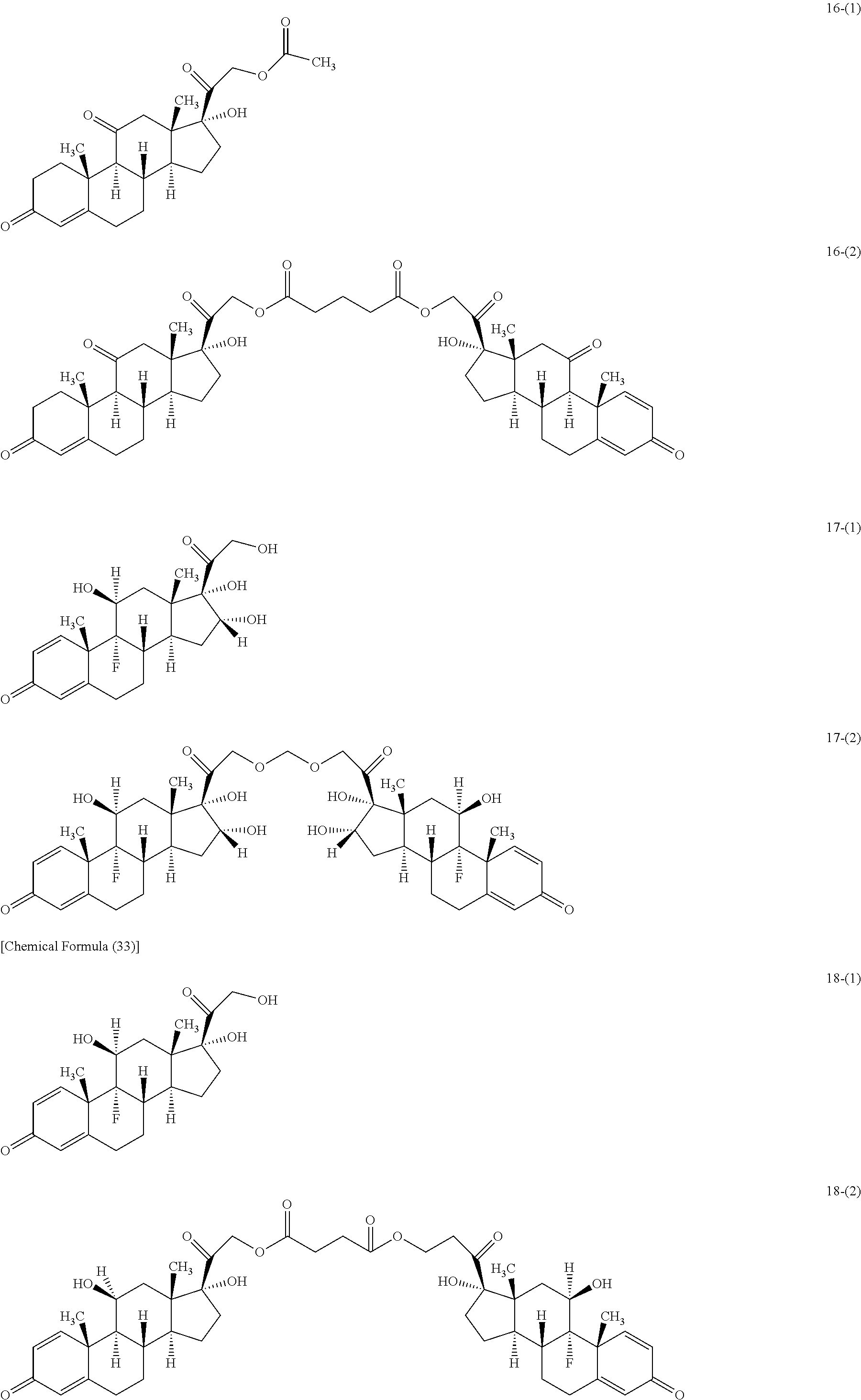

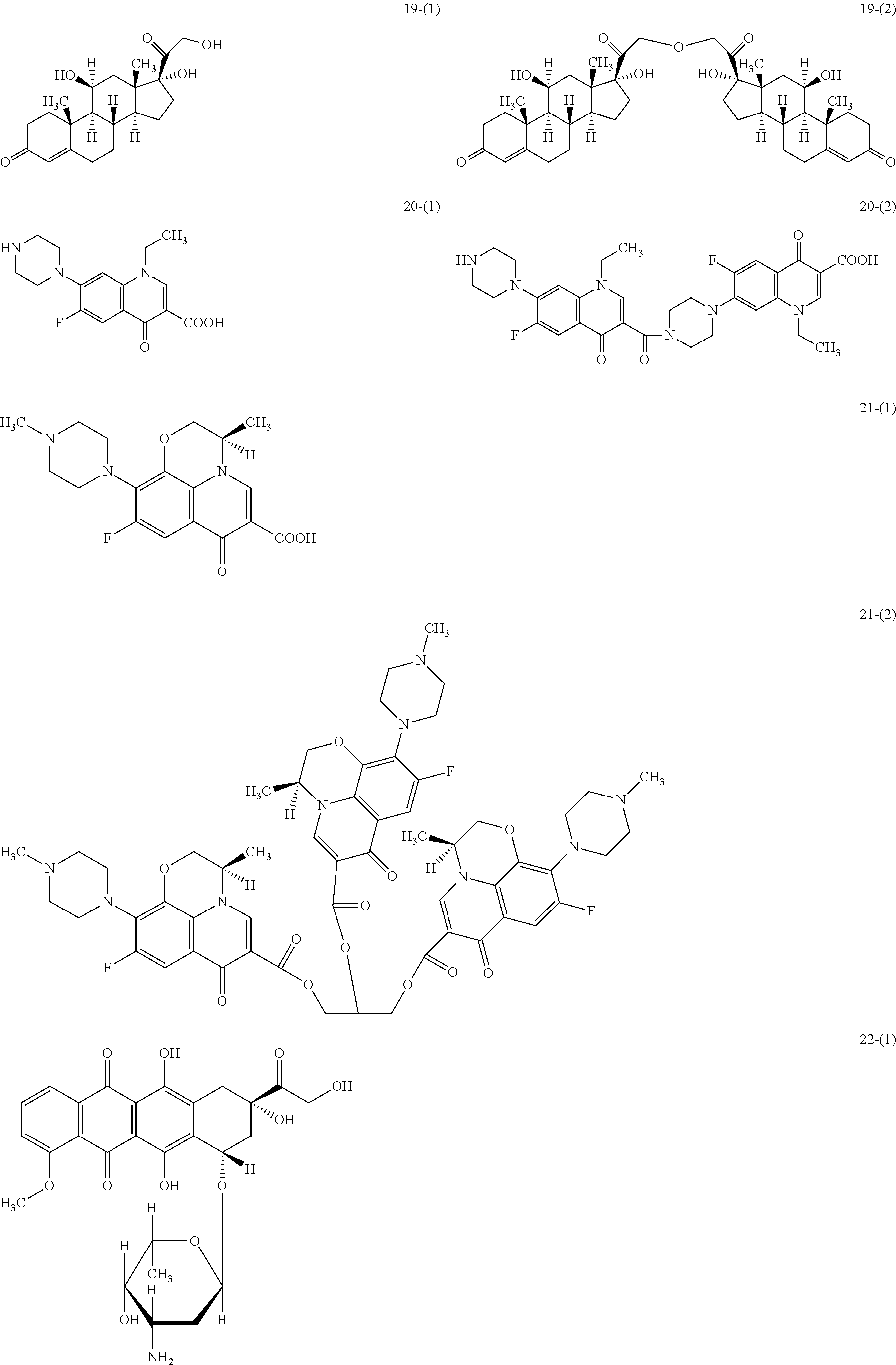

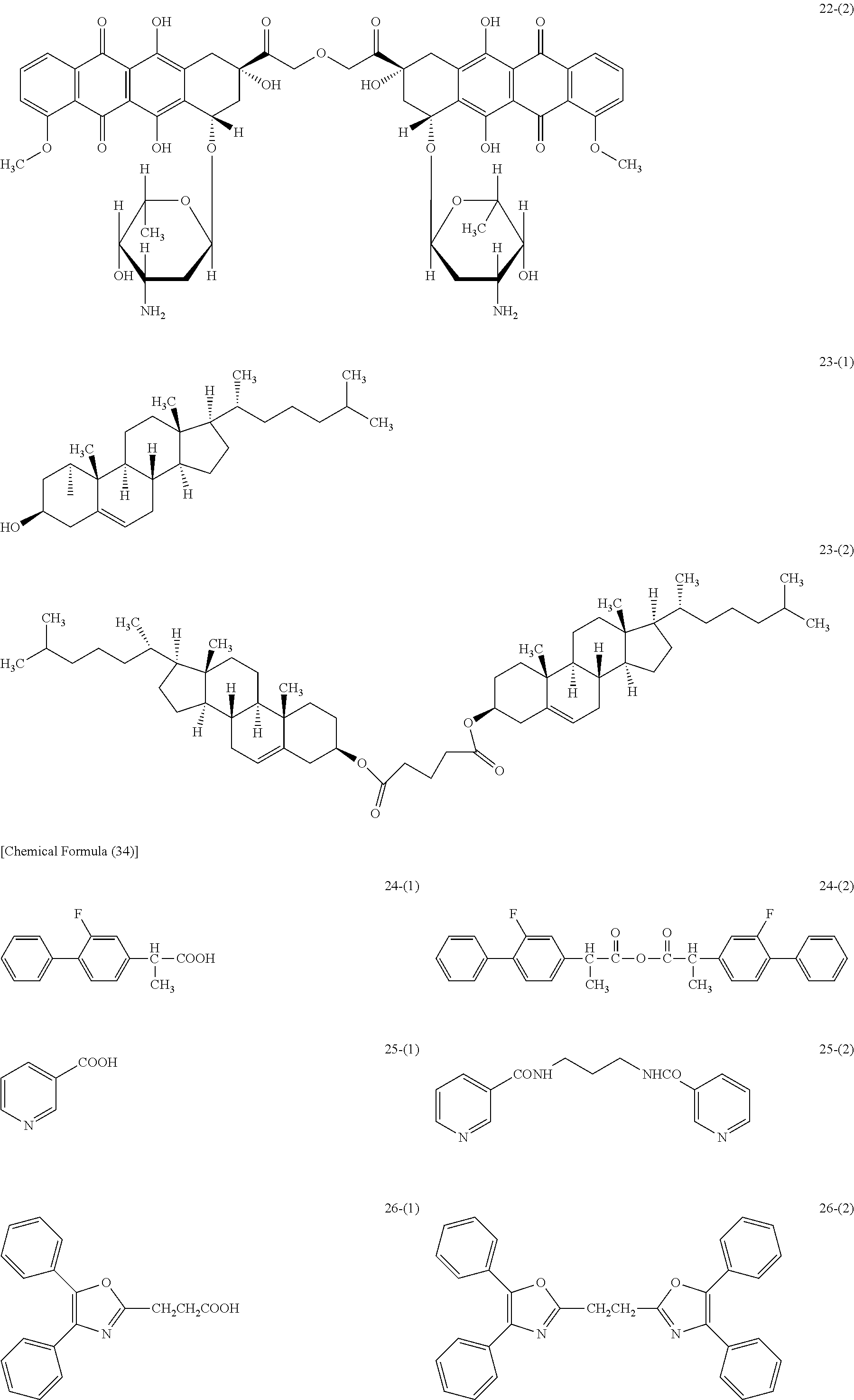

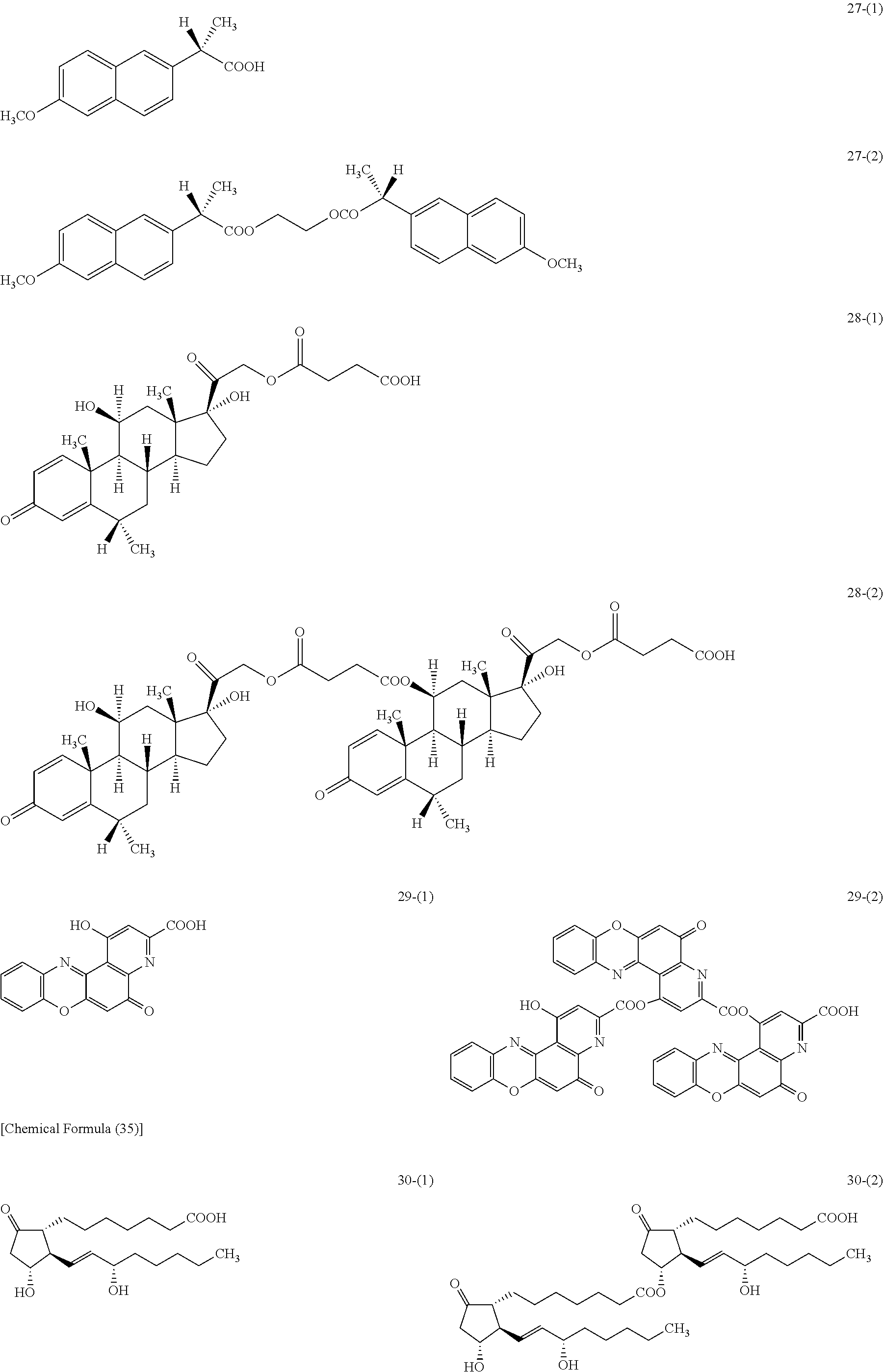

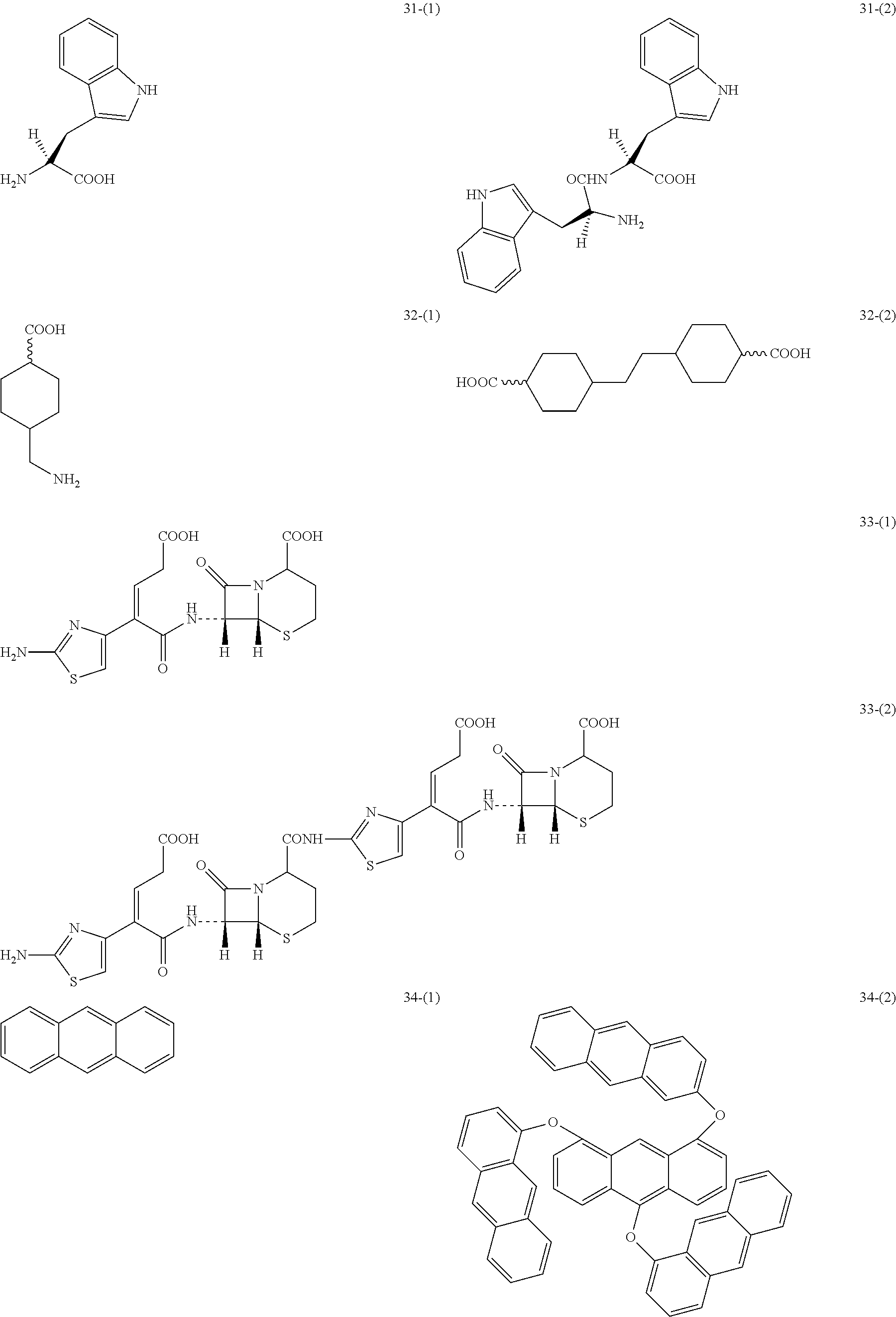

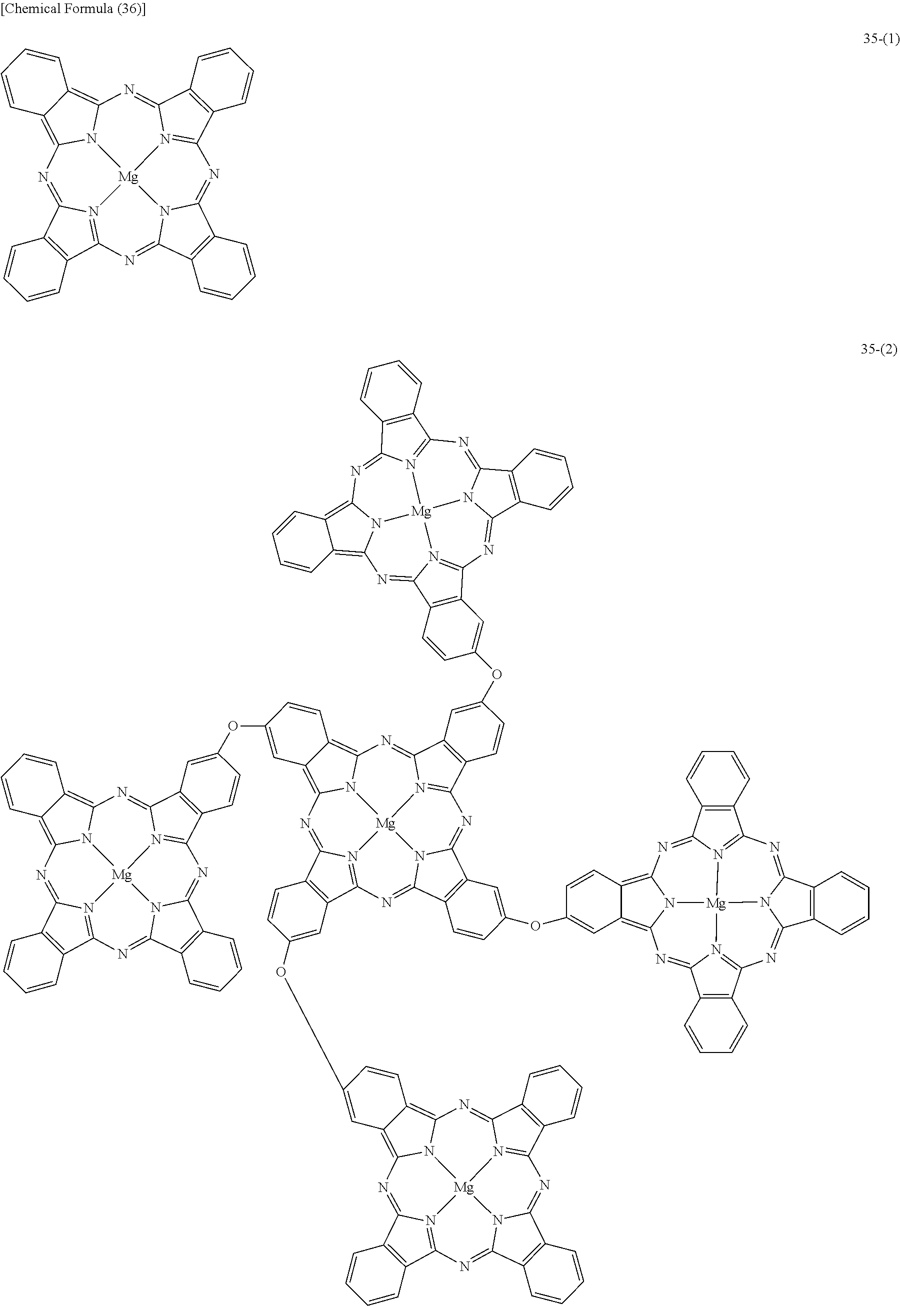

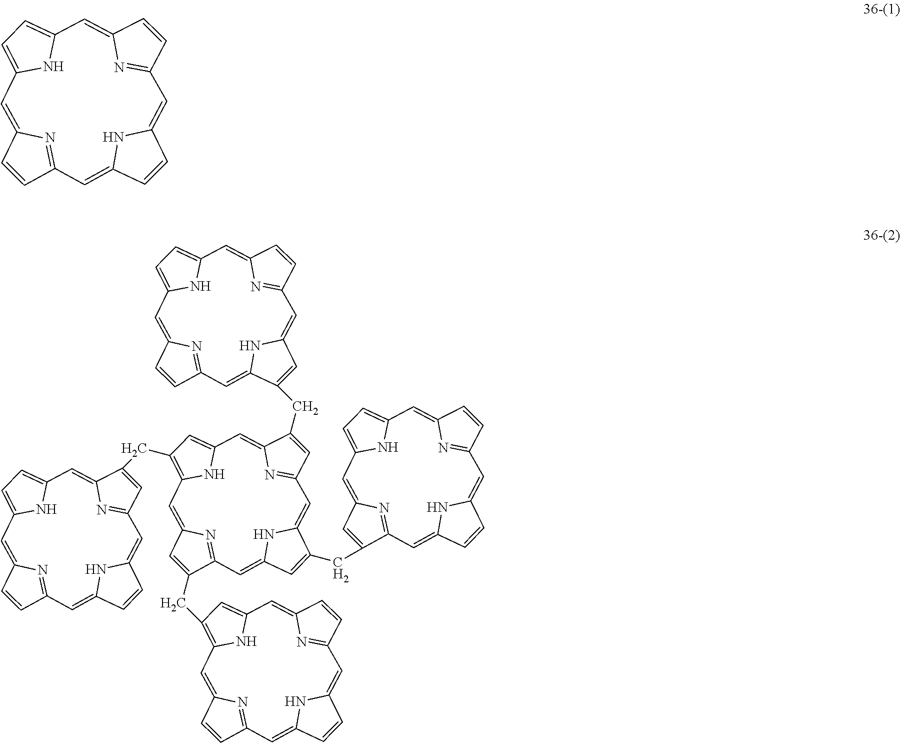

In manufacturing organic particles of the present invention, the compound represented by the general formula (6) or general formula (7) (hereinafter, simply referred to collectively as compound represented by the general formula (6)) and crystal nucleating agent, first, are dissolved in organic solvent which is a good solvent. As long as an organic solvent can dissolve the compound represented by general formula (6) and crystal nucleating agent, such organic solvent can be appropriately selected. As specific organic solvent, depending on the compound to be dissolved and poor solvent to be mentioned later, exemplified are ketones solvent such as acetone, methyl ethyl ketone, etc.; cyclic ethers solvent such as tetrahydrofuran, dioxane, etc.; nitriles solvent such as acetonitrile, etc.; alcohols solvent such as methanol, ethanol, isopropanol, etc.; polar amides solvent such as N,N-dimethylacetamide, N,N-dimethylformamide, N-methylpyrrolidone, etc.; sulfoxides solvent such as dimethylsulfoxide, diethylsulfoxide, etc.; phenols solvent such as phenol, o-, m-, or p-cresol, xylenol, halogenated phenol, catechol, etc.; aprotic polar solvent such as hexamethylphosphoramide, γ-butyrolactone, etc., and these may be used alone or as a mixture thereof.

Since the dissolved concentration of the compound represented by general formula (6) in organic solvent is a major factor affecting the particle size of the microparticles to be formed, the concentration is appropriately changed depending on the desired particle size. The solution concentration is usually adjusted to 0.1˜15% by mass, and when molecular weight is high, the solution is preferably adjusted to around 0.5% by mass.

The crystal nucleating agent to be added together with the compound represented by general formula (6) can't perform fully its function when its addition amount is too little, on the contrary, when too much, a problem as impurities in the particle is raised. Therefore, preferable addition amount of the crystal nucleating agent is, based on molar concentration of the compound represented by general formula (6), an amount wherein total amount of partial structure A possessed by the crystal nucleating agent is 0.0001˜50 mol %, preferably 0.01˜15 mol %. The total amount of partial structure A possessed by the crystal nucleating agent here is a value obtained by multiplying the amount of the crystal nucleating agent by the number of partial structure A contained in the crystal nucleating agent molecule, and for example, when the crystal nucleating agent is the compound represented by general formula (8), the value is that obtained by multiplying the amount of compound represented by general formula (8) by (m′(n′+1)).

The organic solvent solution of the compound represented by the above-mentioned general formula (6) and the crystal nucleating agent (hereinafter, simply referred to as organic solvent solution) is poured into a solvent which is a poor solvent for the organic compound represented by general formula (6) and the crystal nucleating agent and is miscible with the organic solvent used for the organic solvent solution (hereinafter, simply referred to as poor solvent). In the poor solvent, the crystal nucleating agent is deposited earlier than the compound represented by general formula (6) to form numerous fine particles, which work as crystal core and the compound represented by general formula (6) is crystallized. Since by the presence of crystal core caused by the crystal nucleating agent, the crystallization of the compound represented by general formula (6) is completed more rapidly than the case when the crystal nucleating agent is not used, fine particles of the compound represented by general formula (6) having small particle size and narrow particle size distribution can be obtained. The obtained fine particles have a fully small particle size, thus they can be obtained in a state of stable dispersion in poor solvent.

The poor solvent is a solvent wherein the compound represented by general formula (6) and the crystal nucleating agent can be deposited when the above-mentioned organic solvent solution is added. In addition to the solvent exemplified above as organic solvent, examples of the poor solvent include water; aliphatic hydrocarbons such as hexane and heptane; alicyclic hydrocarbons such as decalin and cyclohexane; aromatic hydrocarbons such as benzene, toluene and xylene; and a mixed solvent of 2 or more of these. The poor solvent differs depending on the structure of the compound represented by general formula (6), thus it is appropriately altered according to the fine particles to be produced and purpose. For example, if it is desired to produce fine particles suspended in water of the compound represented by general formula (6), water is used as poor solvent.

A surfactant can be placed into poor solvent so as to improve the dispersion stability of organic particles to be obtained. When a surfactant is used, its amount is not limited particularly, but when too much, it adversely affects the formation of particles, thus usually about 0.1% by mass is used.

The amount of poor solvent that the organic solvent solution is poured into is more than ten times based on the volume of the organic solvent solution. Although there is no upper limit, from the viewpoint of workability and economy for particle recovery, the amount is preferred to be not too much. From the viewpoint of deposition ability and workability of particles, an amount of around 100 times is generally used.

At the time of pouring, the poor solvent is preferred to be kept stirring in order to uniform the particle size of organic particles to be formed. The stirring rate is not particularly limited, but is preferably 100˜3000 rpm.

The temperature of the poor solvent at the time of pouring is not particularly limited as long as the solvent maintains the liquid state, but when the temperature of the poor solvent is changed, the particle size is also changed, therefore by keeping the temperature of poor solvent constant, the particle size of organic particles to be obtained can be controlled. For example, when a poor solvent is used at a temperature below 5° C., the particle size of organic particles tends to increase. Usually, in the range of −20° C. to 60° C., the temperature is controlled depending on the desired particle size.

The pouring should preferably be conducted rapidly and over a short time in order to prevent the variation in character of particle. In the laboratory, usually a syringe is used.

By the above-mentioned operation, usually fine particles having particle size of 50˜10000 nm and small particle size distribution can be obtained in a state of suspension in poor solvent. The particle size can be appropriately adjusted by controlling the temperature of poor solvent, kind of crystal nucleating agent, addition amount thereof and the like. The obtained organic particles can be used in the suspended state as a pigment ink, a suspending agent, and the like, further by carrying out solid-liquid separation operation such as filtration, they can be isolated as powders.

Thus, the present invention is described in reference to the most practical and preferable embodiments at this time, but the present invention is not limited to the embodiments disclosed in this specification, and appropriate changes may be made without departing from the scope or spirits of the invention read by the claims and whole specification, so it must be understood that pharmaceutical multimeric particles and manufacturing method thereof accompanying such changes are included in the technical scope of the present invention.

### Example 1

### Synthesis of SN-38 dimer (Compound 18-(b)) (succinic acid bis(7-ethylcamptothecin-10-yl)ester)

### <Esterification>

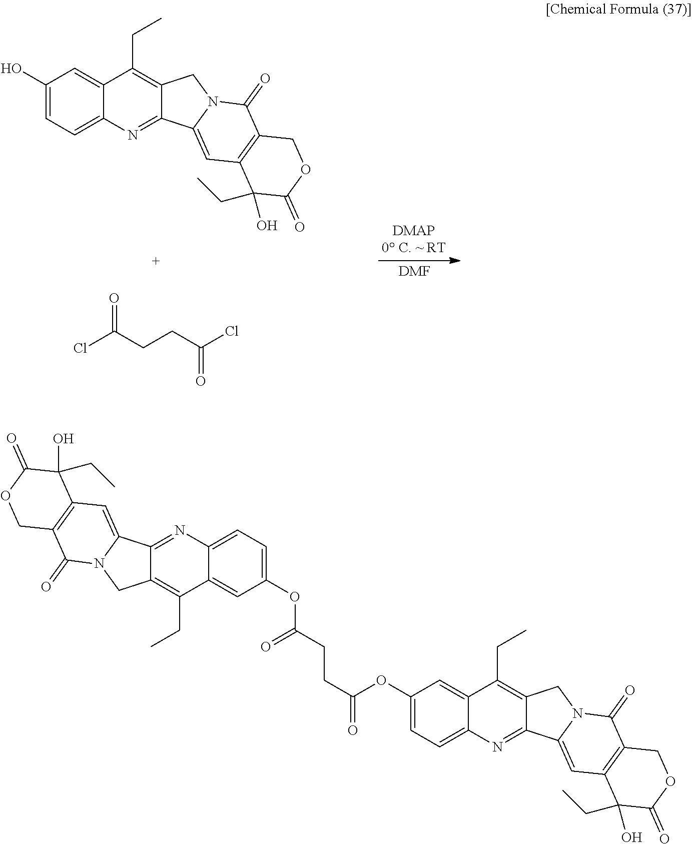

SN-38 (Compound 18-(a)) (10 mg, 0.025 mmol) and 4-dimethylaminopyridine (DMAP: 7 mg, 0.058 mmol) were placed in two-necked eggplant flask, and after nitrogen substitution, 1 ml of dehydrated N,N-dimethylformamide (DMF) was added, followed by stirring for 30 minutes. After this eggplant flask is cooled in ice to 0° C., succinyl dichloride (2.6 μl, 0.023 mmol) was added, and stirred for 4 hours with gradually warming to room temperature.

The stirred solution was transferred to separatory funnel, ethyl acetate was added, and washed more than 2 times with aqueous ammonium chloride solution. The solution was separated into aqueous phase and organic phase, each placed in Erlenmeyer flask, and the aqueous phase was confirmed with thin-layer chromatography (TLC) and the organic phase was filtered through absorbent cotton after dried over magnesium sulfate. The solvent of the solution was completely distilled away under reduced pressure, and the resulting crystals were washed with chloroform/methanol=1/5 on a drain grating under suction filtration. The color of the purified crystals is white-purple, and yield is 55% for ester dimer of SN-38 (Compound 18-(b)).

¹HNMR (400 MHz, DMSO, δ): 0.87 (t, J=7.4 Hz, 6H), 1.26 (t, J=7.2 Hz, 6H), 1.86 (s, 4H), 3.15 (s, 8H), 5.34 (s, 3H), 5.43 (s, 4H), 6.53 (s, 2H), 7.35 (s, 2H), 7.73 (s, 2H)

MS (ESI): m/z=867 (M+H⁺)

### Example 2

### Synthesis of SN-38 dimer (Compound 18-(c)) (butylene bis(7-ethylcamptothecin-10-yl)amide)

### <Urethane Bond>

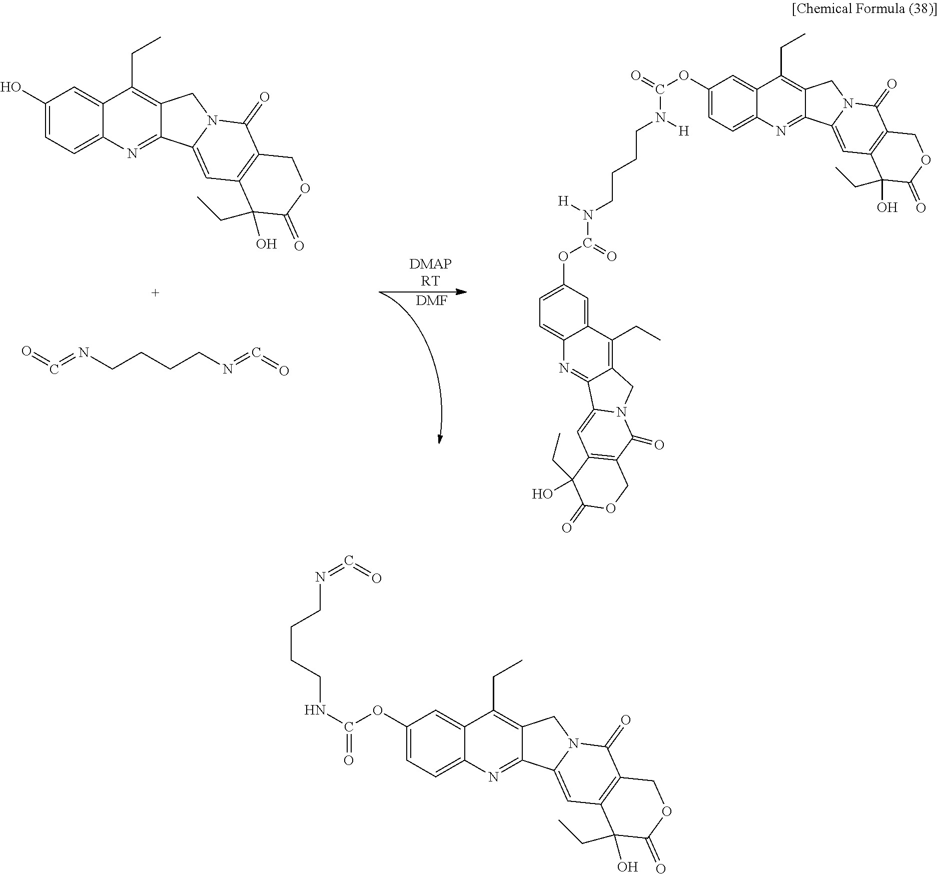

SN-38 20 mg (0.051 mmol) and DMAP (0.116 mmol) were placed in two-necked eggplant flask, and after nitrogen substitution, 1 ml of dehydrated DMF and tetramethylene diisocyanate 3 μl (0.023 mmol) were added, followed by stirring at room temperature overnight. The stirred solution was transferred to separatory funnel, ethyl acetate was added, and washed several times with aqueous ammonium chloride solution. The solution was separated into aqueous phase and organic phase, each placed in Erlenmeyer flask, and the aqueous phase was confirmed with thin-layer chromatography (TLC) and the organic phase was filtered through absorbent cotton after dried over magnesium sulfate. Minimum amount of silica which was adsorbed to the filtrate was added, and the solvent was completely distilled away under reduced pressure, then purified by silica gel column chromatography with chloroform/methanol=20/1 as developing solvent. The color of the purified crystals was yellow, and the yield of dimer of SN-38 and tetramethylene diisocyanate (Compound 18-(c)) was 42%.

¹HNMR (dimer: 400 MHz, DMSO, δ): 0.87 (t, J=6.8 Hz, 6H), 1.26 (t, J=7.3 Hz, 6H), 1.59 (s, 4H), 1.86 (q, J=7.3 Hz, 4H), 3.14 (q, J=8.3 Hz, 4H) [symbol] 3.32 (t, J=21.9 Hz, 4H), 5.29 (s, 4H), 5.42 (s, 4H), 6.51 (s, 2H), 7.30 (s, 2H), 7.66 (q, J=11.7 Hz, 2H), 7.92 (s, 2H), 8.01 (s, 2H), 8.15 (d, J=9.8 Hz, 2H)

MS (ESI: dimer): m/z=924.8 (M+H⁺)

### Example 3

### Synthesis of SN-38 dimer (Compound 18-(d)) (bis(7-ethylcamptothecin-10-oxy)2-buten ether)

### <Ether>

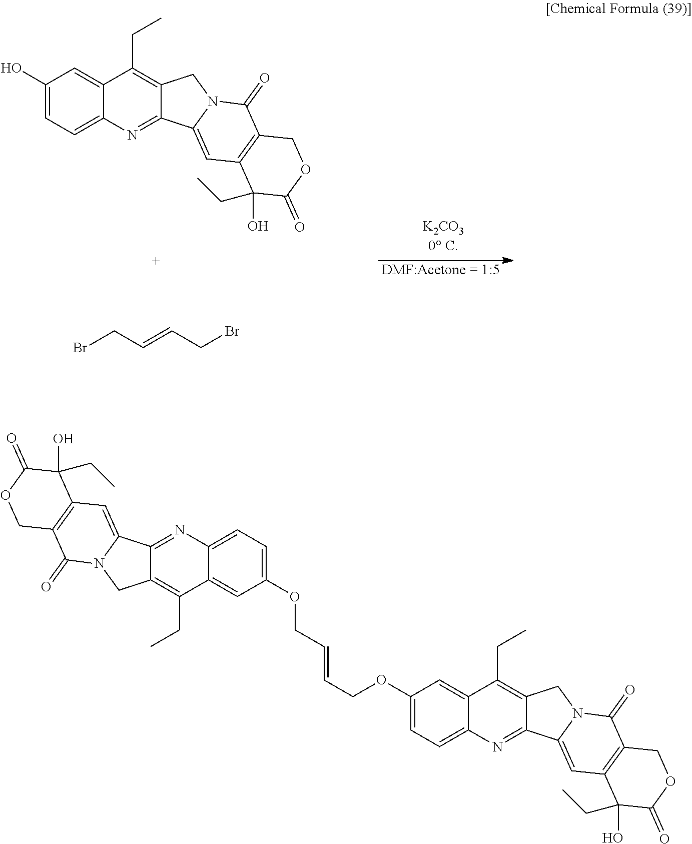

SN-38 (20 mg, 0.051 mmol) was placed in two-necked eggplant flask, and after nitrogen substitution, 0.3 ml of dehydrated DMF was added and stirred for 10 minutes to completely dissolve SN-38. 1,4-Dibromo-2-butene (5.4 mg, 0.025 mmol) was added thereto and stirred to dissolve, then dehydrated acetone 1.5 ml and potassium carbonate (18 mg, 0.127 mmol) were added, and stirred for two night.

The stirred solution was transferred to separatory funnel, ethyl acetate was added, and washed several times with aqueous ammonium chloride solution. The solution was separated into aqueous phase and organic phase, each placed in Erlenmeyer flask, and the aqueous phase was confirmed with thin-layer chromatography (TLC) and the organic phase was filtered through absorbent cotton after dried over magnesium sulfate. Then minimum amount of silica which was adsorbed to the filtrate was added, and the solvent was completely distilled away under reduced pressure. The residue was purified by silica gel column chromatography with chloroform/methanol=10/1 as developing solvent. The color of the purified crystals was pale yellow, and the yield of dimer of SN-38 and 1,4-dibromo-2-butene (Compound 18-(d)) was 23%.

¹HNMR (400 MHz, DMSO, δ): 0.85 (t, J=9.8 Hz, 6H), 1.16 (t, J=7.3 Hz, 6H), 2.02 (q, J=13 Hz, 4H), 2.69 (q, J=11.7 Hz, 4H), 4.01 (s, 4H), 4.58 (d, J=8.2 Hz, 4H), 5.30 (s, 4H), 5.41 (s, 2H), 6.03 (s, 2H) [symbol] 7.26 (s, 2H), 7.52 (s, 4H), 7.94 (s, 2H).

### Example 4

### Synthesis of Betamethasone Valerate Dimer (Compound 19-(b)) Using Esterification

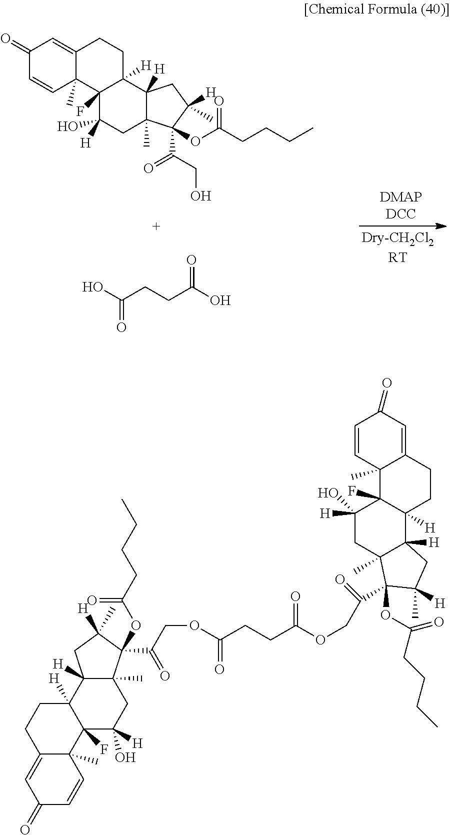 [SMILES: CCCCC(=O)O[C@]1(C(=O)CO)[C@@H](C)C[C@H]2[C@@H]3CCC4=CC(=O)C=C[C@]4(C)[C@@]3(F)[C@@H](O)C[C@@]21C ; O=C(O)CCC(=O)O ; CCCCC(=O)O[C@]1(C(=O)COC(=O)CCC(=O)OCC(=O)[C@@]2(OC(=O)CCCC)[C@@H](C)C[C@H]3[C@@H]4CCC5=CC(=O)C=C[C@]5(C)[C@@]4(F)[C@@H](O)C[C@@]32C)[C@@H](C)C[C@H]2[C@@H]3CCC4=CC(=O)C=C[C@]4(C)[C@@]3(F)[C@@H](O)C[C@@]21C]

Succinic acid (36 mg, 0.286 mmol), 4-dimethylaminopyridine (DMAP: 174 mg, 1.431 mmol) and N,N-dicyclohexylcarbodiimide (DCC: 132 mg, 0.629 mmol) were placed in two-necked eggplant flask, substituted with nitrogen gas, dehydrated dichloromethane (0.1M, 6 ml) were added, and stirred for 30 minutes. Then, betamethasone valerate (300 mg, 0.629 mmol) was added thereto, and stirred overnight. The stirred solution was filtered through Celite to remove the precipitates of urea, and the solvent of the filtrate was completely distilled away under reduced pressure. Chloroform was added thereto to dissolve the products, minimum amount of silica which was adsorbed to the solution was added, and the solvent was distilled away under reduced pressure. Further the silica was dried to be a dry powder by completely removing the remaining solvent using vacuum line, and purified by silica gel column chromatography using ethyl acetate as developing solvent. The color of the purified crystals was white, and the yield of dimer (Compound 19-(b)) was 53%.

¹HNMR (400 MHz, DMSO, δ): 0.91 (t, J=7.3 Hz, 6H), 0.93 (d, J=9.8 Hz, 6H), 1.26 (ddd, J=13.6 Hz, 4H), 1.33 (d, J=7.8 Hz, 6H), 1.35 (q, J=7.8 Hz, 6H), 1.56 (d, J=8.8 Hz, 6H), 1.58 (s, 2H), 1.60 (q, J=5.8 Hz, 4H), 1.62 (d, J=5.8 Hz, 2H), 1.64 (q, J=9.8 Hz, 4H), 1.99 (q, J=10.2 Hz, 2H), 2.12 (t, J=8.8 Hz, 2H), 2.34 (t, J=7.8 Hz, 4H), 2.41 (t, 8.8 Hz, 4H), 2.49 (q, J=6.8 Hz, 2H), 2.63 (s, 2H), 2.78 (t, 5.9 Hz, 4H), 4.40 (t, 7.8 Hz, 2H), 4.84 (s, 4H), 6.14 (s, 2H), 6.34 (s, 2H), 6.36 (s, 2H)

MS (ESI): m/z=1035 (M+H⁺) [symbol] 1057 (M+H⁺+Na⁺)

### Example 5

### How to Make Nanoparticles

### <Ester Bond of SN-38>

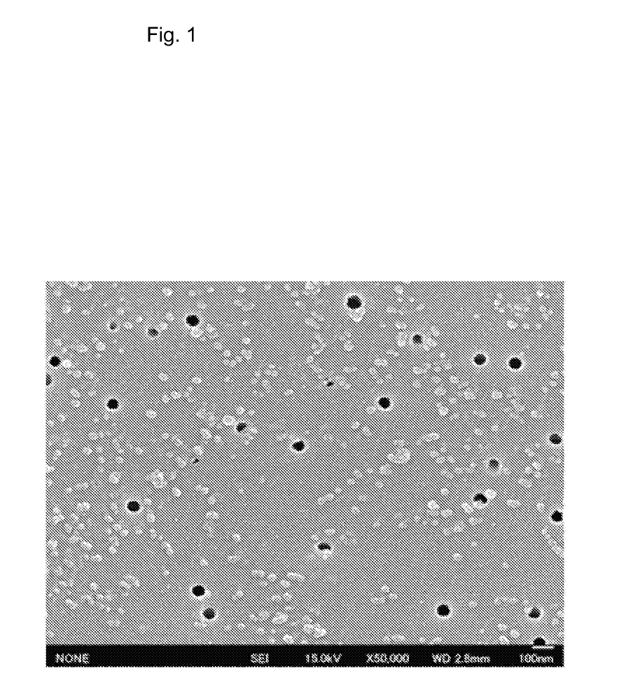

Dimer of SN-38 and succinic acid was weighed out to be 5 mM, and dissolved in DMSO 150 μl. Using water as poor solvent, 100 μl of the DMSO solution was injected into water 10 ml with syringe at room temperature, thus aqueous dispersion solution of dimer nanoparticles was prepared. Final concentration of the aqueous dispersion solution was 0.05 mM. The particle size was 50 nm (FIG. 1).

### Example 6

### How to Make Nanoparticles

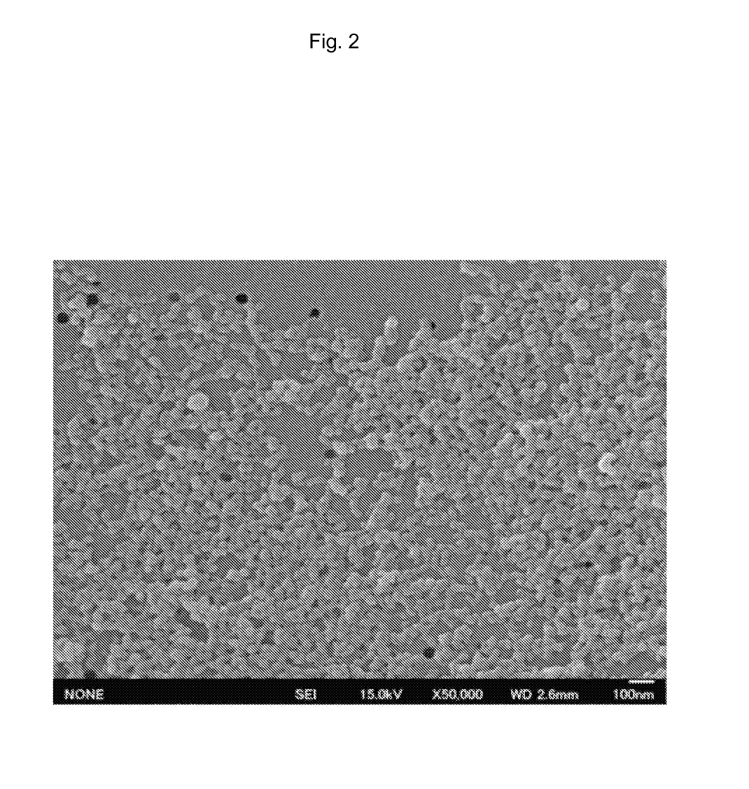

Dimer of SN-38 and tetramethylene diisocyanate was weighed out to be 5 mM, and dissolved in 150 μl of DMSO. Using water as poor solvent, 100 μl of the DMSO solution was injected into water 10 ml with syringe at room temperature, thus aqueous dispersion solution of dimer nanoparticles was prepared. Final concentration of the aqueous dispersion solution was 0.05 mM. The particle size was 50 nm (FIG. 2).

### Example 7

### How to Make Nanoparticles

Dimer of betamethasone valerate and succinic acid was weighed out to be 5 mM, and dissolved in 150 μl of DMSO. Using water as poor solvent, 100 μl of the DMSO solution was injected into water 10 ml with syringe at room temperature, thus aqueous dispersion solution of dimer nanoparticles was prepared. Final concentration of the aqueous dispersion solution was 0.05 mM. The particle size was 150 nm (FIG. 2).

### Example 8

### In Vitro Anticancer Activity of Various SN-38 Dimer Nanoparticles

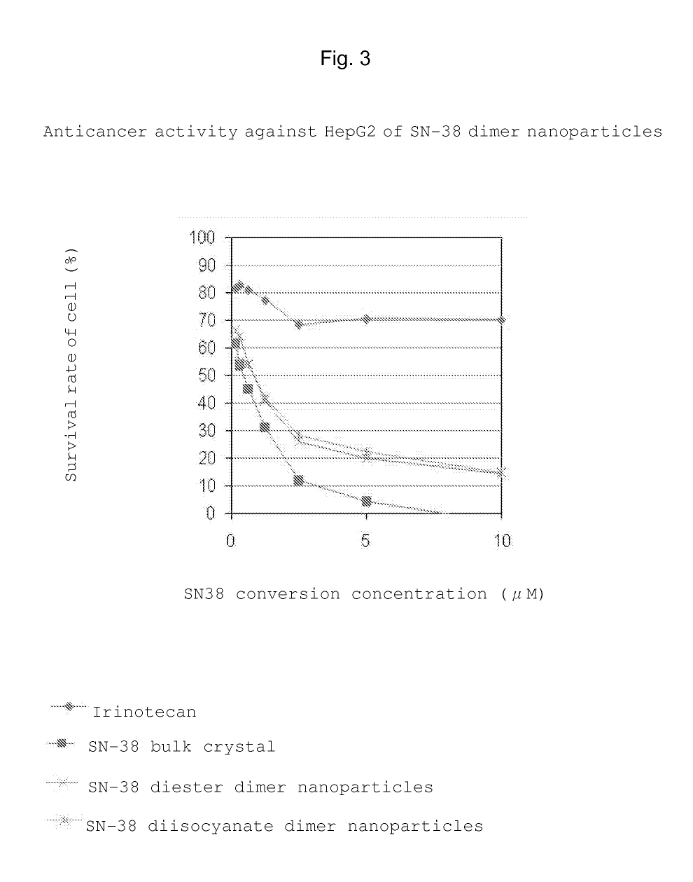

Human liver cancer cell lines HepG2 was seeded on 96-well plate in 2×10⁴ cells/well. Next day, irinotecan, SN-38 bulk crystal, SN-38 diester dimer nanoparticle, and SN-38 diisocyanate dimer nanoparticle were added to the HepG2 cell culture in an amount to be 0.15625, 0.3125, 0.625, 1.25, 2.5, 5 or 10 μM in terms of SN-38 monomer. After cultivating for 48 hours, the cell viability was measured by a colorimetric method using Cell Counting Kit-8 (manufactured by DOJINDO) and microplate reader (manufactured by BIO-RAD). As the result, under the present condition, irinotecan showed almost no anticancer activity, whereas various SN-38 samples showed remarkable anticancer activity. In addition, it became obvious that when SN-38 was dimerized via ester bond or urethane bond, or further nanoparticulated, the anticancer activity thereof can be retained (FIG. 3).

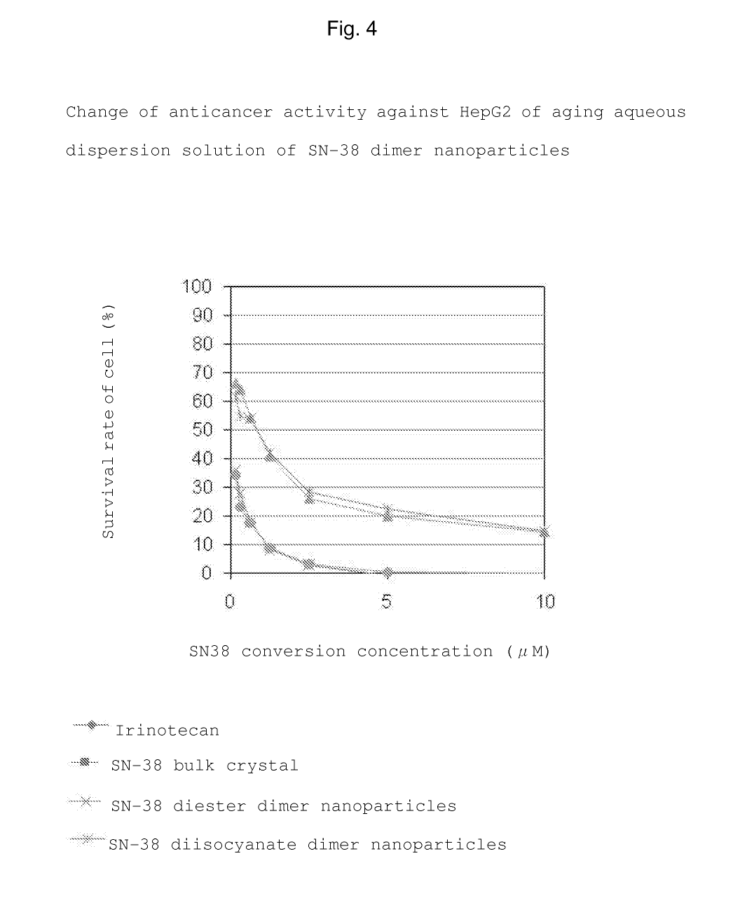

Further, two kinds of aqueous dispersion solution of SN-38 dimer nanoparticles were left at room temperature for about one week, thus prepared aging sample. It was revealed from the study about aging effect as an indicator of anticancer activity against HepG2 cell that in both of the two kinds of SN-38 dimer, the anticancer activity is increased by aging (FIG. 4).

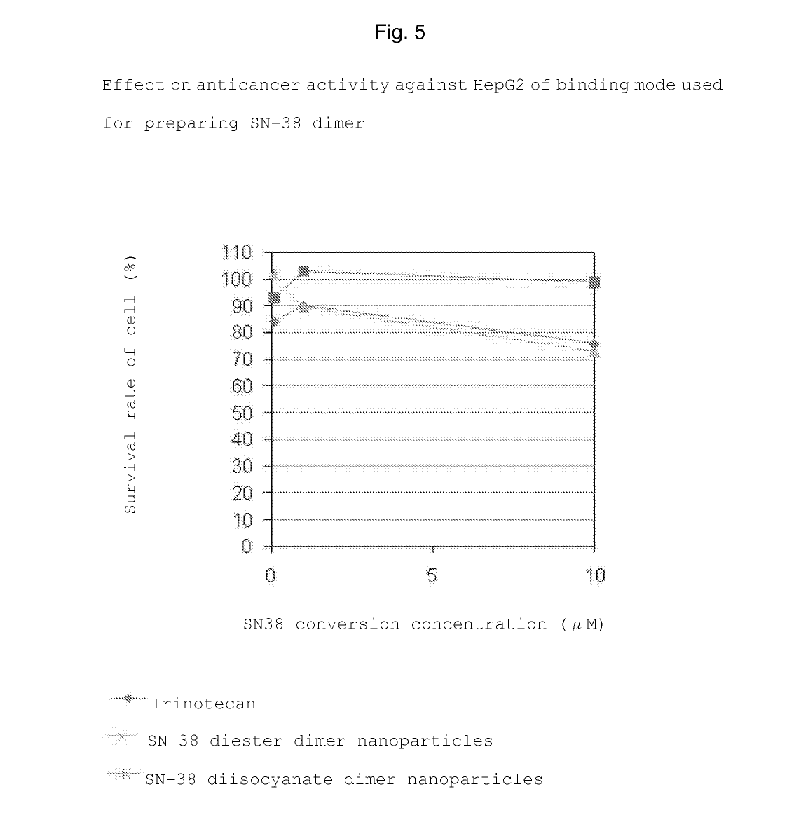

In FIG. 3 and FIG. 4, there was no difference in the anticancer activity against HepG2 between the binding modes included in the SN-38 dimer nanoparticles, namely, ester bond and urethane bond. That is, both bonds are expected to be decomposed at about the same speed in the solution, and SN-38 monomers are formed. To confirm this, SN-38 is dimerized with low degradable ether bond, further subjected to aging, then studied the anticancer activity against HepG2. The treatment concentration at this time was made 0.1, 1, 10 μM in terms of SN-38. As the result, the anticancer activity of the SN-38 diether dimer nanoparticles almost disappeared, and also the aging effect was little (FIG. 5). The above results suggested that the anticancer activity of the SN-38 dimer nanoparticles depends on the decomposition rate of the dimer. On the contrary, it suggests that the anticancer activity of the SN-38 dimer nanoparticle can be controlled by the binding mode used for dimerization, which is considered to be useful for adjusting the medicinal effect expression profile after in vivo administration.

1. Pharmaceutical dimeric particles, that are characterized by being obtained by pouring a solution of the compound represented by general formula (I) dissolved in a water-miscible organic solvent into water.
[Chemical Formula (4)]
D-R-L-R-D (I)
wherein, D represents an anticancer agent, L represents a group selected from a group consisting of an optionally substituted alkyl, an optionally substituted cycloalkyl, an optionally substituted alkenyl, an optionally substituted alkynyl and an optionally substituted aryl, R represents a group selected from a group consisting of —C(═O)—, —C(═O)O—, —C(═O)NR1-, —C(═O)OC(═O)—, —C(═O)OC(═O)O—, —O—, —NHR1-, —S(═O)—, —SO2-, —SO(NR1)-, —SO2NR1, —P(═O)(OR1)O—, —P(═O)(NR)O— and alkylene, R1 represents a hydrogen, alkyl, an optionally substituted cycloalkyl or an optionally substituted aryl.

2. The pharmaceutical dimeric particles according to claim 1, wherein D is a camptothecin derivative, L is an optionally substituted alkyl or an optionally substituted alkenyl, R is —C(═O)—, —C(═O)NR1- or alkylene, and R1 is a hydrogen.

3. The pharmaceutical dimeric particles according to claim 2, wherein D is SN-38.

4. Pharmaceutical multimeric particles, wherein a pharmaceutical multimer is characterized by being formed by intermolecular dehydration condensation between a of plural drugs represented by general formula (1), and being hydrolyzed in vivo to plural drugs represented by the above-mentioned general formula (1), and said pharmaceutical multimeric particles are characterized by being obtained by pouring a solution of the pharmaceutical multimer dissolved in a water-miscible organic solvent into water.
[Chemical Formula (1)]
Aa)ₖ (1)
wherein, in the general formula (1), A is a k-valent organic group, k is an integer of 2 or more, and a is a monovalent substituent. k of a may be the same or different each other.

5. Pharmaceutical multimeric particles of a drug represented by general formula (2), that are characterized by being obtained by pouring a solution of the compound represented by the following general formula (3) dissolved in a water-miscible organic solvent into water. 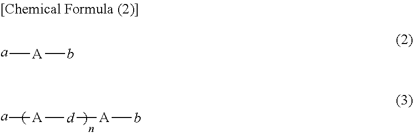
wherein, in the general formula (2) and general formula (3), A is a divalent organic group, a and b are each a substituent selected from hydroxyl group, amino group, carboxyl group, thiol group and hydrogen atom (provided that limited to the hydrogen atom on secondary amine). In the general formula (3), n is an integer of 1 or more, and d is a divalent substituent wherein by in vivo hydrolysis of d, the compound represented by the general formula (3) is converted to n+1 of drugs represented by the general formula (2).

6. Pharmaceutical multimeric particles of a drug represented by general formula (4), that are characterized by being obtained by pouring a solution of the compound represented by general formula (5) dissolved in a water-miscible organic solvent into water.
[Chemical Formula (3)]
a-A (4)
Bd-A)ₘ (5)
wherein, in the general formula (4) and general formula (5), A is a monovalent organic group. In the general formula (4), a is a substituent selected from hydroxyl group, amino group, carboxyl group, thiol group and hydrogen atom (provided that, limited to the hydrogen atom on secondary amine). In the general formula (5), m is an integer of 2 or more, B is a co-valent substituent, and d is a divalent substituent wherein by in vivo hydrolysis of d, the compound represented by the general formula (5) is converted to the compound represented by B(-f)m wherein f is a substituent selected from hydroxyl group, amino group, carboxyl group and thiol group, and m of drug represented by the general formula (4).

7. The pharmaceutical multimeric particles according to claim 6, wherein the compound represented by B(-f)m mentioned above is characterized by any one of succinic acid, fumaric acid, tartaric acid, malic acid, ascorbic acid, citric acid, lactic acid, amino acid, maleic acid, malonic acid, adipic acid, triethanolamine, trometamol and glycerin.

8. The pharmaceutical multimeric particles according to claim 6, which are characterized in that the above mentioned d is any one of groups consisting of —NHCO—, —CONH—, —COO— and —OCO—.

9. The pharmaceutical multimeric particles according to claim 1, which are characterized in that the drug is a poorly water-soluble drug having solubility of 1.0 g/L or less in 25° C. water.

10. A pharmaceutical dimer having the following structural formula.

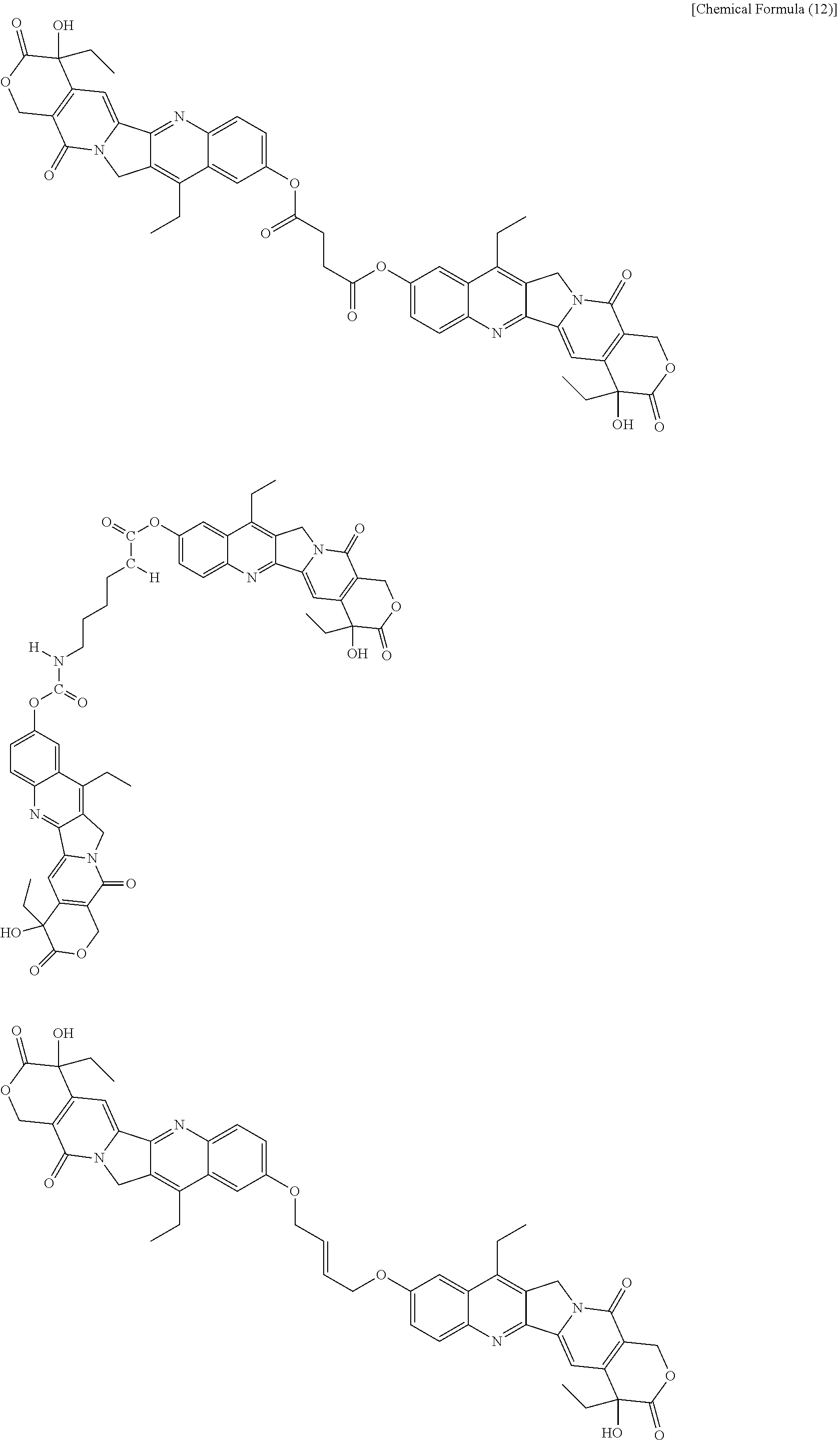 [SMILES: CCc1c2c(nc3ccc(OC(=O)CCC(=O)Oc4ccc5nc6c(c(CC)c5c4)Cn4c-6cc5c(c4=O)COC(=O)C5(O)CC)cc13)-c1cc3c(c(=O)n1C2)COC(=O)C3(O)CC ; CCc1c2c(nc3ccc(OC(=O)CCCCCNC(=O)Oc4ccc5nc6c(c(CC)c5c4)Cn4c-6cc5c(c4=O)COC(=O)C5(O)CC)cc13)-c1cc3c(c(=O)n1C2)COC(=O)C3(O)CC ; CCc1c2c(nc3ccc(OC/C=C/COc4ccc5nc6c(c(CC)c5c4)Cn4c-6cc5c(c4=O)COC(=O)C5(O)CC)cc13)-c1cc3c(c(=O)n1C2)COC(=O)C3(O)CC]

11. Manufacturing method of the pharmaceutical dimeric particles according to claim 1, which is characterized by pouring a solution of the compound represented by general formula (I) dissolved in a water-miscible organic solvent into water.

12. A manufacturing method of pharmaceutical multimeric particles, which is characterized by pouring a solution of pharmaceutical multimer dissolved in a water-miscible organic solvent into water, and said pharmaceutical multimer is characterized by being formed by intermolecular condensation between a of plural drugs represented by the general formula (1), and being converted in the living body to plural drugs represented by the general formula (1).
[Chemical Formula (1)]
Aa)ₖ (1)
wherein, in the general formula (1), A is a k-valent organic group, k is an integer of 2 or more, and a is a monovalent substituent. k of a may be the same or different each other.

13. A manufacturing method of pharmaceutical multimeric particles, which is characterized by pouring a solution of pharmaceutical multimer having a structure represented by the general formula (5) dissolved in a water-miscible organic solvent into water.
